SEQUENCE LISTING (19 sequences)

SEQ ID NO: 1 - protein, 120 aa, Artificial Sequence
  Description of Artificial Sequence Synthetic polypeptide
  HCVR
EVQLVESGGGLVQPGGSLRLSCAASGFTFNNHEMNWVRQAPGKGLEWVSYISSSGNTVTYADFLKGRFTISRDNAKNSLFLQMNSLRDEDTAVYYCARDHLSGTSPLSYWGQGTLVTVSS

SEQ ID NO: 2 - protein, 8 aa, Artificial Sequence
  Description of Artificial Sequence Synthetic peptide
  HCDR1
GFTFNNHE

SEQ ID NO: 3 - protein, 8 aa, Artificial Sequence
  Description of Artificial Sequence Synthetic peptide
  HCDR2
ISSSGNTV

SEQ ID NO: 4 - protein, 13 aa, Artificial Sequence
  Description of Artificial Sequence Synthetic peptide
  HCDR3
ARDHLSGTSPLSY

SEQ ID NO: 5 - protein, 107 aa, Artificial Sequence
  Description of Artificial Sequence Synthetic polypeptide
  LCVR
DIQMTQSPSSLSASVGDRVTITCQASQDINNYLNWFQQKPGKAPKLLIFDASNLETGVPSRFSGSGSGTDFTFTISSLQPEDIATYFCQQYENLPYTFGQGTKLEIK

SEQ ID NO: 6 - protein, 6 aa, Artificial Sequence
  Description of Artificial Sequence Synthetic peptide
  LCDR1
QDINNY

SEQ ID NO: 7 - protein, 3 aa, Artificial Sequence
  Description of Artificial Sequence Synthetic peptide
  LCDR2
DAS

SEQ ID NO: 8 - protein, 9 aa, Artificial Sequence
  Description of Artificial Sequence Synthetic peptide
  LCDR3
QQYENLPYT

SEQ ID NO: 9 - protein, 413 aa, Artificial Sequence
  Description of Artificial Sequence Synthetic polypeptide
  hANGPTL8-mFc aa 1-177 amino acids 22-198 of NP_061157.3 aa 178-413 GPG linker and mouse IgG2a Fc tag
APMGGPELAQHEELTLLFHGTLQLGQALNGVYRTTEGRLTKARNSLGLYGRTIELLGQEVSRGRDAAQELRASLLETQMEEDILQLQAEATAEVLGEVAQAQKVLRDSVQRLEVQLRSAWLGPAYREFEVLKAHADKQSHILWALTGHVQRQRREMVAQQHRLRQIQERLHTAALPAGPGEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 10 - protein, 126 aa, Artificial Sequence
  Description of Artificial Sequence Synthetic polypeptide
  Evinacumab HCVR
EVQLVESGGGVIQPGGSLRLSCAASGFTFDDYAMNWVRQGPGKGLEWVSAISGDGGSTYYADSVKGRFTISRDNSKNSLYLQMNSLRAEDTAFFYCAKDLRNTIFGVVIPDAFDIWGQGTMVTVSS

SEQ ID NO: 11 - protein, 8 aa, Artificial Sequence
  Description of Artificial Sequence Synthetic peptide
  Evinacumab HCDR1
GFTFDDYA

SEQ ID NO: 12 - protein, 8 aa, Artificial Sequence
  Description of Artificial Sequence Synthetic peptide
  Evinacumab HCDR2
ISGDGGST

SEQ ID NO: 13 - protein, 19 aa, Artificial Sequence
  Description of Artificial Sequence Synthetic peptide
  Evinacumab HCDR3
AKDLRNTIFGVVIPDAFDI

SEQ ID NO: 14 - protein, 108 aa, Artificial Sequence
  Description of Artificial Sequence Synthetic polypeptide
  Evinacumab LCVR
DIQMTQSPSTLSASVGDRVTITCRASQSIRSWLAWYQQKPGKAPKLLIYKASSLESGVPSRFSGSGSGTEFTLTISSLQPDDFATYYCQQYNSYSYTFGQGTKLEIKR

SEQ ID NO: 15 - protein, 6 aa, Artificial Sequence
  Description of Artificial Sequence Synthetic peptide
  Evinacumab LCDR1
QSIRSW

SEQ ID NO: 16 - protein, 3 aa, Artificial Sequence
  Description of Artificial Sequence Synthetic peptide
  Evinacumab LCDR2
KAS

SEQ ID NO: 17 - protein, 9 aa, Artificial Sequence
  Description of Artificial Sequence Synthetic peptide
  Evinacumab LCDR3
QQYNSYSYT

SEQ ID NO: 18 - protein, 460 aa, Artificial Sequence
  Description of Artificial Sequence Synthetic polypeptide
  hANGPTL3
MFTIKLLLFIVPLVISSRIDQDNSSFDSLSPEPKSRFAMLDDVKILANGLLQLGHGLKDFVHKTKGQINDIFQKLNIFDQSFYDLSLQTSEIKEEEKELRRTTYKLQVKNEEVKNMSLELNSKLESLLEEKILLQQKVKYLEEQLTNLIQNQPETPEHPEVTSLKTFVEKQDNSIKDLLQTVEDQYKQLNQQHSQIKEIENQLRRTSIQEPTEISLSSKPRAPRTTPFLQLNEIRNVKHDGIPAECTTIYNRGEHTSGMYAIRPSNSQVFHVYCDVISGSPWTLIQHRIDGSQNFNETWENYKYGFGRLDGEFWLGLEKIYSIVKQSNYVLRIELEDWKDNKHYIEYSFYLGNHETNYTLHLVAITGNVPNAIPENKDLVFSTWDHKAKGHFNCPEGYSGGWWWHDECGENNLNGKYNKPRAKSKPERRRGLSWKSQNGRLYSIKSTKMLIHPTDSESFE

SEQ ID NO: 19 - protein, 198 aa, Homo sapiens
MPVPALCLLWALAMVTRPASAAPMGGPELAQHEELTLLFHGTLQLGQALNGVYRTTEGRLTKARNSLGLYGRTIELLGQEVSRGRDAAQELRASLLETQMEEDILQLQAEATAEVLGEVAQAQKVLRDSVQRLEVQLRSAWLGPAYREFEVLKAHADKQSHILWALTGHVQRQRREMVAQQHRLRQIQERLHTAALPA

(19) United States
(12) Patent Application Publication
(10) Pub. No.: US 2017/0291937 A1
(43) Pub. Date: Oct. 12, 2017

(54) Methods for Treating Hyperlipidemia with an ANGPTL8 Inhibitor and an ANGPTL3 Inhibitor

(71) Applicant: Regeneron Pharmaceuticals, Inc., Tarrytown, NY (US)

(72) Inventors: Jesper Gromada, Scarsdale, NY (US); Viktoria Gusarova, Pleasantville, NY (US); Andrew J. Murphy, Croton-on-Hudson, NY (US)

(21) Appl. No.: 15/482,499

(22) Filed: Apr. 7, 2017

Related U.S. Application Data
(60) Provisional application No. 62/319,980, filed on Apr. 8, 2016.
(60) Provisional application No. 62/453,110, filed on Feb. 1, 2017.

(51) Int. Cl. C07K 16/22; A61K 9/00

(52) U.S. Cl. CPC C07K 16/22; A61K 9/0019; A61K 2039/505

(57) ABSTRACT

The present invention provides methods for treating patients suffering from hyperlipidaemia, wherein the patient is non-responsive to, inadequately controlled by, or intolerant to treatment with a standard lipid modifying therapy. The methods of the invention provide for lowering at least one lipid parameter in the patient by administering a therapeutically effective amount of an antibody or antigen-binding fragment thereof that specifically binds to angiopoietin-like protein 8 (ANGPTL8) in combination with a therapeutically effective amount of an antibody that specifically binds to angiopoietin-like protein 3 (ANGPTL3). The combination of an anti-ANGPTL8 antibody with an anti-ANGPTL3 antibody is useful in treating diseases such as hypercholesterolemia, including familial hypercholesterolemia (FH), both heFH and hoFH, as well as hyperlipidaemia, hyperlipoproteinemia and dyslipidemia, including hypertriglyceridemia, chylomicronemia, and to prevent or treat diseases or disorders, for which abnormal lipid metabolism is a risk factor, such as cardiovascular diseases.

### CROSS-REFERENCE TO RELATED APPLICATIONS

This application claims the benefit under 35 U.S.C. §119(e) of U.S. provisional application Nos. 62/453,110, filed on Feb. 1, 2017 and 62/319,980, filed on Apr. 8, 2016. The disclosure of the aforementioned patent application is herein incorporated by reference in its entirety.

### SEQUENCE STATEMENT

The instant application contains a Sequence Listing, which has been submitted electronically in ASCII format and is hereby incorporated by reference in its entirety. Said ASCII copy, created on Apr. 7, 2017, is named Sequence list_25.TXT and is 16,560 bytes in size.

### FIELD OF THE INVENTION

The present invention relates to the field of therapeutic treatments of diseases and disorders, which are associated with elevated levels of lipids and lipoproteins. More specifically, the invention relates to the use of an angiopoietin-like protein 8 (ANGPTL8) inhibitor in combination with an inhibitor of angiopoietin-like protein 3 (ANGPTL3) to treat patients with hypercholesterolemia and related conditions.

### BACKGROUND

Hyperlipidaemia is a general term that encompasses diseases and disorders characterized by or associated with elevated levels of lipids and/or lipoproteins in the blood. Hyperlipidaemia include hypercholesterolemia, hypertriglyceridemia, combined hyperlipidaemia, and elevated lipoprotein a (Lp(a)). A particular prevalent form of hyperlipidaemia in many populations is hypercholesterolemia.

Hypercholesterolemia, particularly an increase in low-density lipoprotein (LDL) cholesterol (LDL-C) levels, constitutes a major risk for the development of atherosclerosis and coronary heart disease (CHD) (Sharrett et al., 2001, Circulation 104:1108-1113). Low-density lipoprotein cholesterol is identified as the primary target of cholesterol lowering therapy and is accepted as a valid surrogate therapeutic endpoint. Numerous studies have demonstrated that reducing LDL-C levels reduces the risk of CHD with a strong direct relationship between LDL-C levels and CHD events; for each 1 mmol/L (˜40 mg/dL) reduction in LDL-C, cardiovascular disease (CVD) mortality and morbidity is lowered by 22%. Greater reductions in LDL-C produce greater reduction in events, and comparative data of intensive versus standard statin treatment suggest that the lower the LDL-C level, the greater the benefit in patients at very high cardiovascular (CV) risk.

Familial hypercholesterolemia (FH) is an inherited disorder of lipid metabolism that predisposes a person to premature severe cardiovascular disease (CVD) (Kolansky et al., (2008), Am J Cardiology,102(11):1438-1443). FH can be either an autosomal dominant or an autosomal recessive disease that results from mutations in the low density lipoprotein receptor (LDLR), or in at least 3 different genes that code for proteins involved in hepatic clearance of LDL-C can cause FH. Examples of such defects include mutations in the gene coding for the LDL receptor (LDLR) that removes LDL-C from the circulation, and in the gene for apolipoprotein (Apo) B, which is the major protein of the LDL particle. In all cases, FH is characterized by an accumulation of LDL-C in the plasma from birth and subsequent development of tendon xanthomas, xanthelasmas, atheromata, and CVD. FH can be classified as either heterozygous FH (heFH) or homozygous FH (hoFH) depending on whether the individual has a genetic defect in one (heterozygous) or both (homozygous) copies of the implicated gene.

Current LDL-C-lowering medications include statins, cholesterol absorption inhibitors, fibrates, niacin, and bile acid sequestrants. Statins are a commonly prescribed treatment for LDL-C lowering. However, despite the availability of such lipid-lowering therapies, many high-risk patients fail to reach their guideline target LDL-C level (Gitt et al., 2010, Clin Res Cardiol 99(11):723-733). For patients who are still unable to achieve guideline target level for LDL-C, despite available lipid-modifying therapy (LMT), mechanical removal of LDL-C by lipoprotein apheresis (e.g., LDL apheresis) is sometimes prescribed.

However, patients who are not at LDL-C goal despite receiving an optimized LMT regimen, would greatly benefit from alternative LDL-C lowering therapies, or through use of a combination of therapeutic agents, such as the agents and regimens described herein.

### BRIEF SUMMARY OF THE INVENTION

In a first aspect, the present invention provides for methods of treating patients suffering from hyperlipidaemia by administering a therapeutically effective amount of an inhibitor of angiopoietin-like protein 8 (ANGPTL8) in combination with a therapeutically effective amount of an inhibitor of angiopoietin-like protein 3 (ANGPTL3).

In one embodiment, the hyperlipidaemia is familial hyperlipidaemia or acquired hyperlipidaemia.

In one embodiment, the hyperlipidaemia is selected from the group consisting of hyperlipoproteinemia, hypercholesterolemia, hypertriglyceridemia, or hyper chylomicronemia.

In one embodiment, the familial hyperlipidaemia is hypercholesterolemia selected from the group consisting of heterozygous familial hypercholesterolemia (HeFH) and homozygous familial hypercholesterolemia (HoFH).

In one embodiment, the acquired hyperlipidaemia is the result of excessive drinking of alcohol, obesity, side effects of medications (e.g. hormones or steroids), diabetes, kidney disease, underactive thyroid gland, or pregnancy.

In one embodiment, the ANGPTL8 inhibitor is an antibody, or an antigen-binding fragment thereof, that binds specifically to ANGPTL8.

In one embodiment, the antibody, or antigen-binding fragment thereof that binds specifically to ANGPTL8 comprises the complementary determining regions (CDRs) of a heavy chain variable (HCVR) having the amino acid sequence of SEQ ID NO: 1 and the CDRs of a light chain variable region (LCVR) of SEQ ID NO: 5.

In one embodiment, the antibody, or antigen-binding fragment thereof that binds specifically to ANGPTL8 comprises a heavy chain CDR1 (HCDR1) having the amino acid sequence of SEQ ID NO: 2, a HCDR2 having the amino acid sequence of SEQ ID NO: 3, a HCDR3 having the amino acid sequence of SEQ ID NO: 4, a light chain CDR1 (LCDR1) having the amino acid sequence of SEQ ID NO: 6, a LCDR2 having the amino acid sequence of SEQ ID NO: 7, and a LCDR3 having the amino acid sequence of SEQ ID NO: 8.

In one embodiment, the antibody, or antigen-binding fragment thereof that binds specifically to ANGPTL8 comprises a HCVR having the amino acid sequence of SEQ ID NO: 1 and a LCVR having the amino acid sequence of SEQ ID NO: 5.

In one embodiment, the ANGPTL8 antibody is administered to the patient subcutaneously or intravenously.

In one embodiment, the ANGPTL3 inhibitor is an antibody, or an antigen-binding fragment thereof, that binds specifically to ANGPTL3.

In one embodiment, the ANGPTL3 antibody is evinacumab.

In one embodiment, the antibody, or antigen-binding fragment thereof that binds specifically to ANGPTL3 comprises the complementary determining regions (CDRs) of a heavy chain variable (HCVR) having the amino acid sequence of SEQ ID NO: 10 and the CDRs of a light chain variable region (LCVR) of SEQ ID NO: 14.

In one embodiment, the antibody, or antigen-binding fragment thereof that binds specifically to ANGTL3 comprises a heavy chain CDR1 (HCDR1) having the amino acid sequence of SEQ ID NO: 11, a HCDR2 having the amino acid sequence of SEQ ID NO: 12, a HCDR3 having the amino acid sequence of SEQ ID NO: 13, a light chain CDR1 (LCDR1) having the amino acid sequence of SEQ ID NO: 15, a LCDR2 having the amino acid sequence of SEQ ID NO: 16, and a LCDR3 having the amino acid sequence of SEQ ID NO: 17.

In one embodiment, the antibody, or antigen-binding fragment thereof that binds specifically to ANGPTL3 comprises a HCVR having the amino acid sequence of SEQ ID NO: 10 and a LCVR having the amino acid sequence of SEQ ID NO: 14.

In one embodiment, the ANGPTL3 antibody is administered to the patient subcutaneously or intravenously.

In a second aspect, the invention provides for methods of reducing the level of at least one lipid parameter in a patient suffering from a disorder or condition characterized in part by elevated levels of lipids or lipoproteins, the method comprising administering to the patient a therapeutically effective amount of a combination of an angiopoietin-like protein 8 (ANGPTL8) inhibitor and an inhibitor of angiopoietin-like protein 3 (ANGPTL3).

In one embodiment, the disorder or condition is hyperlipidaemia selected from the group consisting of familial hyperlipidaemia and acquired hyperlipidaemia.

In one embodiment, the disorder or condition is hyperlipidaemia selected from the group consisting of hyperlipoproteinemia, hypercholesterolemia, hypertriglyceridemia, or hyper chylomicronemia.

In one embodiment, the familial hyperlipidaemia is hypercholesterolemia selected from the group consisting of heterozygous familial hypercholesterolemia (HeFH) and homozygous familial hypercholesterolemia (HoFH).

In one embodiment, the acquired hyperlipidaemia is the result of excessive drinking of alcohol, obesity, side effects of medications (e.g. hormones or steroids), diabetes, kidney disease, underactive thyroid gland, or pregnancy.

In one embodiment, the hypertriglyceridemia treatable by the methods of the invention include familial combined hyperlipidaemia, familial dysbetalipoproteinemia, familial hypertriglyceridemia, severe chylomicronemia and familial lipoprotein lipase deficiency.

In one embodiment, the ANGPTL8 inhibitor is an antibody, or an antigen-binding fragment thereof, that binds specifically to ANGPTL8.

In one embodiment, the antibody, or antigen-binding fragment thereof that binds specifically to ANGPTL8 comprises the complementary determining regions (CDRs) of a heavy chain variable (HCVR) having the amino acid sequence of SEQ ID NO: 1 and the CDRs of a light chain variable region (LCVR) of SEQ ID NO: 5.

In one embodiment, the antibody, or antigen-binding fragment thereof that binds specifically to ANGPTL8 comprises a heavy chain CDR1 (HCDR1) having the amino acid sequence of SEQ ID NO: 2, a HCDR2 having the amino acid sequence of SEQ ID NO: 3, a HCDR3 having the amino acid sequence of SEQ ID NO: 4, a light chain CDR1 (LCDR1) having the amino acid sequence of SEQ ID NO: 6, a LCDR2 having the amino acid sequence of SEQ ID NO: 7, and a LCDR3 having the amino acid sequence of SEQ ID NO: 8.

In one embodiment, the antibody, or antigen-binding fragment thereof that binds specifically to ANGPTL8 comprises a HCVR having the amino acid sequence of SEQ ID NO: 1 and a LCVR having the amino acid sequence of SEQ ID NO: 5.

In one embodiment, the ANGPTL8 antibody is administered to the patient subcutaneously or intravenously.

In one embodiment, the ANGPTL3 inhibitor is an antibody, or an antigen-binding fragment thereof, that binds specifically to ANGPTL3.

In one embodiment, the ANGPTL3 antibody is evinacumab.

In one embodiment, the antibody, or antigen-binding fragment thereof that binds specifically to ANGPTL3 comprises the complementary determining regions (CDRs) of a heavy chain variable (HCVR) having the amino acid sequence of SEQ ID NO: 10 and the CDRs of a light chain variable region (LCVR) of SEQ ID NO: 14.

In one embodiment, the antibody, or antigen-binding fragment thereof that binds specifically to ANGTL3 comprises a heavy chain CDR1 (HCDR1) having the amino acid sequence of SEQ ID NO: 11, a HCDR2 having the amino acid sequence of SEQ ID NO: 12, a HCDR3 having the amino acid sequence of SEQ ID NO: 13, a light chain CDR1 (LCDR1) having the amino acid sequence of SEQ ID NO: 15, a LCDR2 having the amino acid sequence of SEQ ID NO: 16, and a LCDR3 having the amino acid sequence of SEQ ID NO: 17.

In one embodiment, the antibody, or antigen-binding fragment thereof that binds specifically to ANGPTL3 comprises a HCVR having the amino acid sequence of SEQ ID NO: 10 and a LCVR having the amino acid sequence of SEQ ID NO: 14.

In one embodiment, the ANGPTL3 antibody is administered to the patient subcutaneously or intravenously.

In one embodiment, the ANGPTL8 inhibitor and the ANGPTL3 inhibitor are administered concurrently or sequentially.

In one embodiment, the ANGPTL8 inhibitor and the ANGPTL3 inhibitor are administered at therapeutically effective concentrations in separate pharmaceutical compositions or are co-formulated in one pharmaceutical composition.

In a third aspect, the present invention provides methods for treating hyperlipidaemia in patients who are non-responsive to, inadequately controlled by, or intolerant to treatment with a standard lipid modifying therapy. The therapeutic methods of the present invention result in a lowering of serum lipoprotein levels to a normal and acceptable range and as such, may act to reduce the risk of development of atherosclerosis, or coronary heart disease.

In one embodiment, the invention provides a method of treating a patient suffering from hypercholesterolemia, wherein the patient is non-responsive to, inadequately controlled by, or intolerant to treatment with a standard lipid modifying therapy, the method comprising treating the patient with a combination of an angiopoietin-like protein 8 (ANGPTL8) inhibitor and an inhibitor of angiopoietin-like protein 3 (ANGPTL3).

In one embodiment, the invention provides administering one or more doses of an ANGPTL8 inhibitor in combination with one or more doses of an ANGPTL3 inhibitor to a patient who is being treated, or has been treated with a standard lipid modifying therapy, but has not responded to such therapy. Administration of a combination of an ANGPTL8 inhibitor with an ANGPTL3 inhibitor to the patient results in lowering the level of at least one lipoprotein in the serum of the patient and consequently reduces or eliminates the need for treatment with the standard lipid lowering therapy by the patient.

In a fourth aspect, the methods of the present invention comprise selecting a patient with hypercholesterolemia who is being treated, or has been treated with a standard lipid lowering therapy and who is non-responsive to, inadequately controlled by, or intolerant to, such therapy and administering one or more doses of a ANGPTL8 antibody in combination with one or more doses of an ANGPTL3 antibody to the patient, thereby lowering the level of at least one lipoprotein in the serum of the patient and consequently replacing the use of the standard lipid modifying therapy with the combination therapy of a ANGPTL8 antibody plus an ANGPTL3 antibody to achieve a target lipoprotein level.

Patients who are treated or treatable by the methods of the present invention include, e.g., patients with hypercholesterolemia, including patients with familial hypercholesterolemia (FH). In certain embodiments, the patients who are treated or treatable by the methods of the present invention are patients who are diagnosed with (or otherwise known to have), homozygous FH (hoFH) or heterozygous FH (heFH), or at risk for developing abnormally high lipid and/or lipoprotein levels associated with homozygous FH (hoFH) or heterozygous FH (heFH).

The present invention also provides pharmaceutical compositions comprising an ANGPTL8 inhibitor and an ANGPTL3 inhibitor for use in treating a patient who is non-responsive to, inadequately controlled by, or intolerant to treatment with a standard lipid modifying therapy, such as a statin. The statin may be selected from the group consisting of atorvastatin (LIPITOR®), pitavastatin (LIVALO®), lovastatin (MEVACOR®), simvastatin (ZOCOR®), pravastatin (PRAVACHOL®) fluvastatin (LESCOL®) and rosuvastatin (CRESTOR®). Other standard lipid lowering agents that may be used in patients suffering from hypercholesterolemia, include, but are not limited to, fibrates, niacin, bile acid sequestrants, ezetimibe (ZETIA®), lomitapide (JUXTAPID®), phytosterols, orlistat (XENICAL®).

Exemplary ANGPTL8 inhibitors, or ANGPTL3 inhibitors that may be used in the context of the methods of the present invention include, e.g., anti-ANGPTL8 or anti-ANGPTL3 antibodies, small molecule inhibitors, and scaffold-based, i.e. ANGPTL8-binding molecules, or ANGPTL3-binding molecules.

In certain embodiments, it is envisioned that the use of the combination of the ANGPTL8 inhibitor with the ANGPTL3 inhibitor may be sufficiently effective at lowering serum lipid and/or lipoprotein levels, such that the dose of the standard lipid modifying therapy may be reduced to eliminate any untoward effects, or it may be eliminated altogether.

In one embodiment, the administration of the ANGPTL8 antibody in combination with the ANGPTL3 antibody results in an additive effect on lowering the blood level of triglycerides and total cholesterol.

In one embodiment, the administration of the ANGPTL8 antibody in combination with the ANGPTL3 antibody results in a synergistic effect on lowering the blood level of triglycerides and total cholesterol.

In one embodiment, the administration of the ANGPTL8 antibody in combination with the ANGPTL3 antibody results in lowering one or more of the following parameters:
(a) a reduction in serum total cholesterol (TC) level; or
(b) a reduction in serum triglycerides (TG);

wherein the reduction of (a), and/or (b) are determined relative to the patient's serum TC level, or serum triglyceride levels prior to, or at the time of initiation of treatment with the combination of the ANGPTL8 inhibitor and the ANGPTL3 inhibitor.

Other embodiments of the present invention will become apparent from a review of the ensuing detailed description.

### BRIEF DESCRIPTION OF THE FIGURES

FIG. 1 shows the effect of H4H1276S (also referred to as evinacumab), an anti-ANGPTL3 antibody, on triglyceride levels in ANGPTL8 knockout (KO) mice or in wild type (WT) mice at day 2 or 8 after administration.

FIG. 2 shows the effect of H4H1276S on total cholesterol levels in ANGPTL8 knockout mice or in wild type mice at day 2 or 8 after administration.

FIG. 3 shows the effect of H4H1276S and H4H15341P (also referred to as “H4H15341”, an anti-ANGPTL8 antibody) on triglyceride levels in humanized ANGPTL8 mice on days 1, 4, 7 and 14 after administration.

FIG. 4 shows the effect of H4H1276S and H4H15341P (an anti-ANGPTL8 antibody) on total cholesterol levels in humanized ANGPTL8 mice on days 1, 4, 7 and 14 after administration.

FIG. 5 shows the results of anti-ANGPTL8 monoclonal antibodies epitope mapping: PEPSCAN ELISA was performed to examine the binding of anti-ANGPTL8 antibodies to 15-amino acid long peptides with 14 amino acid overlap, spanning mature ANGPTL8.

FIGS. 6a, 6b, 6c, 6d, 6e, and 6f show ANGPTL8 antibody reverses ANGPTL3:ANGPTL8-mediated LPL inhibition by steric interference of LPL with ANGPTL8 inhibitory motif. (FIG. 6a) ANGPTL8 amino acid sequence (SEQ ID NO:19) depicting epitopes of eight monoclonal antibodies raised against human ANGPTL8. (FIG. 6b) Serum triglycerides of humanized ANGPTL8 mice, 7 days before (pre-bleed) and 2 days after a single injection of 10 mg/ml of the eight monoclonal antibodies with epitopes shown in (FIG. 6a). (FIG. 6c) LPL activity measured in cell media from HEK283T cells co-transfected with ANGPTL3 and ANGPTL8 were treated with increased concentration of anti-ANGPTL8 blocking antibody (mAb7) or control antibody. (FIG. 6d) AlphaLISA assay to determine interaction between ANGPTL3 and ANGPTL8 co-expressed in CHO-K1 cells with no or increasing concentrations of either anti-ANGPTL8 blocking antibody (mAb7) or control antibody. (FIG. 6e) Scheme of the TR-FRET assay with donor dye at the N-terminus of ANGPTL8, near its inhibitory motif, and blocking and non-blocking antibodies labeled with acceptor dye. (FIG. 6f) Energy transfer measured by TR-FRET assay between the N-terminus labeled ANGPTL8 and control antibody, anti-ANGPTL8 blocking antibody (mAb7), or non-blocking anti-ANGPTL8 (mAb4) (n=3; ***p<0.0001). The experiment was repeated 3 times with the similar result. Abbreviations: Bio, biotin; SA, streptavidin, mAb, monoclonal antibody; D, donor dye; A, acceptor dye.

### DETAILED DESCRIPTION

Before the present invention is described, it is to be understood that this invention is not limited to particular methods and experimental conditions described, as such methods and conditions may vary. It is also to be understood that the terminology used herein is for the purpose of describing particular embodiments only, and is not intended to be limiting, since the scope of the present invention will be limited only by the appended claims.

Unless defined otherwise, all technical and scientific terms used herein have the same meaning as commonly understood by one of ordinary skill in the art to which this invention belongs. As used herein, the term “about,” when used in reference to a particular recited numerical value, means that the value may vary from the recited value by no more than 1%. For example, as used herein, the expression “about 100” includes 99 and 101 and all values in between (e.g., 99.1, 99.2, 99.3, 99.4, etc.).

Although any methods and materials similar or equivalent to those described herein can be used in the practice of the present invention, the preferred methods and materials are now described. All publications mentioned herein are incorporated herein by reference to describe in their entirety.

Methods for Treating Hyperlipidaemia

The present invention relates generally to methods and compositions for reducing lipoprotein levels, particularly triglycerides and total cholesterol, in patients suffering from hyperlipidaemia, by administering a combination of an inhibitor of ANGPTL8 with an inhibitor of ANGPTL3 to a patient in need of such therapy. In certain embodiments, the combination of inhibitors is used to treat hypercholesterolemia, or hypertriglyceridemia, in patients who are non-responsive to, inadequately controlled by, or intolerant to standard lipid modifying therapies (e.g. a statin). In certain embodiments of the invention, treatment with an ANGPTL8 inhibitor in combination with an ANGPTL3 inhibitor may serve to lower the levels of lipoproteins in these patients to an acceptable range, thereby lowering their risk for development of atherosclerosis, stroke and other cardiovascular diseases. In certain embodiments, the methods described may be used to treat patients suffering from hypercholesterolemia, including heterozygous familial hypercholesterolemia (heFH) and/or homozygous familial hypercholesterolemia (hoFH), in the event that these patients are non-responsive to, inadequately controlled by, or intolerant to standard lipid modifying therapies. In certain embodiment, the methods described may be used to treat patients suffering from, or at risk for developing atherosclerosis, coronary artery disease, diabetes, obesity, pancreatitis, or metabolic syndrome.

As used herein, the term “lipoprotein” means a biomolecular particle containing both protein and lipid. Examples of lipoproteins include, e.g., low density lipoprotein (LDL), high-density lipoprotein (HDL), very low density lipoprotein (VLDL), intermediate density lipoprotein (IDL), and lipoprotein (a) (Lp(a)).

The present invention, according to certain embodiments, includes methods for treating patients who are non-responsive to, inadequately controlled by, or intolerant to standard lipid modifying therapy. As used herein, a particular patient who is “non-responsive to, inadequately controlled by, or intolerant to, standard lipid modifying therapy” is determined by a physician, physician's assistant, diagnostician, or other medical professional on the basis of the level of one or more lipoproteins (e.g., LDL-C and/or non-HDL-C) measured or otherwise detected in the serum of the patient after treatment with the standard lipid modifying agent. The physician, physician's assistant, diagnostician, or other medical professional can also determine if the patient is intolerant to standard lipid modifying therapies based on the side effect profile of the standard lipid modifying therapies, which the patient may experience, including, but not limited to, muscle aches, tenderness or weakness (myalgia), headache, skin flushing, difficulty sleeping, abdominal cramping, bloating, diarrhea, constipation, rash, nausea, or vomiting. A patient who is non-responsive to, inadequately controlled by, or intolerant to standard lipid modifying therapy may also be determined or influenced by other factors such as the patient's family history, medical background, current therapeutic treatment status, as well as generally accepted or prevailing lipoprotein targets adopted by national medical associations and physicians' groups. For example, in certain contexts, if a patient is undergoing therapy with a standard lipid modifying agent, and exhibits an LDL-C level of greater than or equal to about 70 mg/dL, this indicates that the patient is “non-responsive to, or inadequately controlled by, or intolerant to standard lipid modifying therapy” and may benefit by treatment using the therapies described herein. In other contexts, if a patient is undergoing therapy with a standard lipid modifying agent, and exhibits an LDL-C level of greater than or equal to about 100 mg/dL, this indicates that the patient is “non-responsive to, inadequately controlled by, or intolerant to standard lipid modifying therapy” and may benefit by treatment using the therapies described herein. In certain contexts, if a patient is undergoing therapy with a standard lipid modifying agent, and exhibits an LDL-C level of greater than or equal to about 150 mg/dL, 200 mg/dL, 250 mg/dL, 300 mg/dL, 400 mg/dL or higher, this indicates that the patient is “non-responsive to, inadequately controlled by, or intolerant to standard lipid modifying therapy” and may benefit by treatment using the therapies described herein. In yet other contexts, whether or not a particular percentage reduction in LDL-C or non-HDL-C level is met, relative to the patient's LDL-C or non-HDL-C level at a particular start point (“baseline”) can be used to determine whether the patient has responded to standard lipid modifying therapy or whether that patient is in need of further treatment using the methods and agents of the present invention. For instance, a reduction in LDL-C or non-HDL-C of less than 50% (e.g., less than 40%, less than 35%, less than 30%, less than 25%, etc.) from baseline may signify a need for therapy using the methods and agents of the invention.

For example, in certain contexts, if a patient is undergoing therapy with a standard lipid modifying agent, and exhibits a triglyceride (TG) level of greater than or equal to about 150 mg/dL, this indicates that the patient is “non-responsive to, or inadequately controlled by, or intolerant to standard lipid modifying therapy” and may benefit by treatment using the therapies described herein. In other contexts, if a patient is undergoing therapy with a standard lipid modifying agent, and exhibits a TG level of greater than or equal to about 200 mg/dL, this indicates that the patient is “non-responsive to, inadequately controlled by, or intolerant to standard lipid modifying therapy” and may benefit by treatment using the therapies described herein. In certain contexts, if a patient is undergoing therapy with a standard lipid modifying agent, and exhibits a TG level of greater than or equal to about 300 mg/dL, 400 mg/dL, 500 mg/dL, 750 mg/dL, 1000 mg/dL or higher, this indicates that the patient is “non-responsive to, inadequately controlled by, or intolerant to standard lipid modifying therapy” and may benefit by treatment using the therapies described herein.

In yet other contexts, whether or not a particular percentage reduction in TG, TC, LDL-C or non-HDL-C level is met, relative to the patient's TG, TC, LDL-C or non-HDL-C level at a particular start point (“baseline”) can be used to determine whether the patient has responded to standard lipid modifying therapy or whether that patient is in need of further treatment using the methods and agents of the present invention. For instance, a reduction in TG, TC, LDL-C or non-HDL-C of less than 50% (e.g., less than 40%, less than 35%, less than 30%, less than 25%, etc.) from baseline may signify a need for therapy using the methods and agents of the invention.

The present invention, accordingly, includes methods of treatment comprising administration of one or more doses of an ANGPTL8 inhibitor combined with one or more doses of an ANGPTL3 inhibitor to a patient, whereby the patient's post-treatment levels of total cholesterol, TG, LDL-C, and/or non-HDL-C are significantly reduced in numbers. For example, the present invention includes therapeutic methods comprising administering one or more doses of an ANGPTL8 inhibitor and one or more doses of an ANGPTL3 inhibitor to a patient who is undergoing standard lipid modifying therapy, but is non-responsive to such therapy, or is intolerant to such therapy, wherein, after receiving one or more doses of the ANGPTL8 inhibitor and one or more doses of the ANGPTL3 inhibitor, the patient is able to achieve normal levels of total cholesterol, TG, LDL-C, or non-LDL-C. In certain instances, the patient may be taken off of the standard lipid modifying therapy, or the standard lipid modifying therapy may be continued, but may be administered at lower doses and may be used in combination with the ANGPTL8 inhibitor and the ANGPTL3 inhibitor, to achieve and/or maintain a particular target lipoprotein level. Alternatively, the patient may be administered the standard lipid modifying therapy at the normal prescribed dose, but the frequency of administration of the lipid modifying therapy may be reduced if the standard lipid modifying therapy is to be administered in conjunction with the combination of the ANGPTL8 inhibitor and the ANGPTL3 inhibitor. In some instances, the need for treatment with the standard lipid modifying therapy by the patient to achieve and/or maintain a particular target lipoprotein level may be eliminated altogether following administration of one or more doses of the ANGPTL8 inhibitor in conjunction with the ANGPTL3 inhibitor.

According to certain embodiments, the present invention comprises methods for reducing or eliminating the need for standard lipid modifying therapy, wherein the methods comprise selecting a patient with hyperlipidaemia (e.g., hypercholesterolemia, or hypertriglyceridemia) who has been treated with lipid modifying therapy within the last month, the last 2 months, the last 3 months, the last 4 months, the last 5 months, the last 6 months, or for a longer period, and administering one or more doses of a ANGPTL8 inhibitor in combination with an ANGPTL3 inhibitor to the patient. The methods according to this aspect of the invention result in lowering the level of at least one lipoprotein in the serum of the patient, and consequently allow for a reduction or elimination of the need for treatment with the standard lipid modifying therapy by the patient. For example, in certain embodiments of the present invention, following administration of one or more doses of a ANGPTL8 inhibitor in combination with an ANGPTL3 inhibitor, the serum LDL-C level of the patient is reduced to less than a defined level (e.g., less than 100 mg/dL or less than 70 mg/dL), or the total cholesterol is lowered to a defined level (e.g. less than 200 mg/dL, or less than 150 mg/dL), or the TG level is reduced to a defined level (e.g. less than 200 mg/dL, or less than 150 mg/dL).

According to certain embodiments, the patient who is treatable by the methods of the present invention has hypercholesterolemia (e.g., a serum LDL-C concentration of greater than or equal to 70 mg/dL, or a serum LDL-C concentration greater than or equal to 100 mg/dL). According to certain embodiments, the patient who is treatable by the methods of the present invention has hypertriglyceridemia (e.g., a serum TG concentration of greater than or equal to 150 mg/dL, or a serum TG concentration greater than or equal to 200 mg/dL). In certain embodiments, the patient's hypercholesterolemia is inadequately controlled by standard lipid modifying therapy, e.g.

statin therapy. For example, the present invention includes methods for treating a patient who is non-responsive, inadequately controlled by, or intolerant to, therapy with a standard lipid modifying therapy, such as a statin, or who has hypercholesterolemia that is inadequately controlled by a daily dose of a statin selected form the group consisting of atorvastatin (including atorvastatin +ezetimibe), rosuvastatin, cerivastatin, pitavastatin, fluvastatin, lovastatin, simvastatin (including simvastatin+ezetimibe), pravastatin, and combinations thereof. The present invention also includes methods for reducing cholesterol, TG, LDL-C, or non-LDL-C in a patient who has hypercholesterolemia, or hypertriglyceridemia and who exhibits statin intolerance or who otherwise experiences adverse or undesirable reaction(s) to statin therapy (e.g., skeletal muscle pain, aches, weakness or cramping [e.g., myalgia, myopathy, rhabdomyolysis, etc.]).

### Patient Selection

The present invention includes methods and compositions useful for treating patients who are suffering from hyperlipidaemia, who are non-responsive to, inadequately controlled by, or intolerant to, therapy with a standard lipid modifying therapy. The patients who are treatable by the methods of the present invention may also exhibit one or more of additional selection criteria. For example, a patient may be selected for treatment with the methods of the present invention if the patient is diagnosed with or identified as being at risk of developing a hypercholesterolemia condition such as, e.g., heterozygous Familial Hypercholesterolemia (heFH), homozygous Familial Hypercholesterolemia (hoFH), Autosomal Dominant Hypercholesterolemia, autosomal recessive hypercholesterolemia (ARH, e.g., ARH associated with mutations in LDLRAP1), as well as incidences of hypercholesterolemia that are distinct from Familial Hypercholesterolemia (nonFH). Diagnosis of familial hypercholesterolemia (e.g., heFH or hoFH) can be made by genotyping and/or clinical criteria. For patients who are not genotyped, clinical diagnosis may be based on either the Simon Broome criteria with a criteria for definite FH, or the WHO/Dutch Lipid Network criteria with a score>8 points.

According to certain embodiments, the patient may be selected on the basis of having a history of coronary heart disease (CHD). As used herein a “history of CHD” (or “documented history of CHD”) includes one or more of: (i) acute myocardial infarction (MI); (ii) silent MI; (iii) unstable angina; (iv) coronary revascularization procedure (e.g., percutaneous coronary intervention [PCI] or coronary artery bypass graft surgery [CABG]); and/or (v) clinically significant CHD diagnosed by invasive or non-invasive testing (such as coronary angiography, stress test using treadmill, stress echocardiography or nuclear imaging).

According to certain embodiments, the patient may be selected on the basis of having non-coronary heart disease cardiovascular disease (“non-CHD CVD”). As used herein, “non-CHD CVD” includes one or more of: (i) documented previous ischemic stroke with a focal ischemic neurological deficit that persisted more than 24 hours, considered as being of a therothrombotic origin; (ii) peripheral arterial disease; (iii) abdominal aortic aneurysm; (iv) atherosclerotic renal artery stenosis; and/or (v) carotid artery disease (transient ischemic attacks or>50% obstruction of a carotid artery).

According to certain embodiments, the patient may be selected on the basis of having one or more additional risk factors such as, e.g., (i) documented moderate chronic kidney disease (CKD) as defined by 30 ≦eGFR<60 mL/min/1.73 m2 for 3 months or more; (ii) type 1 or type 2 diabetes mellitus with or without target organ damage (e.g., retinopathy, nephropathy, microalbuminuria); (iii) a calculated 10-year fatal CVD risk SCORE≧5% (ESC/EAS Guidelines for the management of dyslipidemias, Conroy et al., 2003, Eur. Heart J. 24:987-1003).

According to certain embodiments, the patient may be selected on the basis of having one or more additional risk factors selected from the group consisting of age (e.g., older than 40, 45, 50, 55, 60, 65, 70, 75, or 80 years), race, national origin, gender (male or female), exercise habits (e.g., regular exerciser, non-exerciser), other preexisting medical conditions (e.g., type-II diabetes, high blood pressure, etc.), and current medication status (e.g., currently taking beta blockers, niacin, ezetimibe, fibrates, omega-3 fatty acids, bile acid resins, etc.).

According to certain embodiments of the present invention, the subject who is treatable by the methods of the invention exhibits an elevated level of one or more inflammatory marker. Any marker of systemic inflammation can be utilized for the purposes of the present invention. Suitable inflammatory markers include, without limitation, C-reactive protein, cytokines (e.g., II-6, IL-8, and/or IL-17), and cellular adhesion molecules (e.g., ICAM-1, ICAM-3, BL-CAM, LFA-2, VCAM-1, NCAM, and PECAM).

According to the present invention, patients may be selected on the basis of a combination of one or more of the foregoing selection criteria or therapeutic characteristics. For example, according to certain embodiments, a patient suitable for treatment with the methods of the present invention, may further be selected on the basis of having heFH or non-FH in combination with: (i) a history of documented CHD, (ii) non-CHD CVD, and/or (iii) diabetes mellitus with target organ damage; such patients may also be selected on the basis of having a serum LDL-C concentration of greater than or equal to 70 mg/dL.

According to certain other embodiments, a patient suitable for treatment with the methods of the present invention, in addition to having hypercholesterolemia that is not adequately controlled by a daily moderate-dose therapeutic statin regimen, may further be selected on the basis of having heFH or non-FH without CHD, or non-CHD CVD, but having either (i) a calculated 10-year fatal CVD risk SCORE≧5%; or (ii) diabetes mellitus without target organ damage; such patients may also be selected on the basis of having a serum LDL-C concentration of greater than or equal to 100 mg/dL.

According to certain embodiments of the present invention, the subject who is treatable by the methods of the invention is a subject who has familial chylomicronemia syndrome (FCS; also known as lipoprotein lipase deficiency).

According to certain embodiments of the present invention, the subject who is treatable by the methods of the invention is a subject who is undergoing, or has recently undergone, lipoprotein apheresis (e.g., within the last six months, within the last 12 weeks, within the last 8 weeks, within the last 6 weeks, within the last 4 weeks, within the last 2 weeks, etc.).

Administration of an ANGPTL8 Inhibitor Plus an ANGPTL3 Inhibitor as Add-On Therapy

The present invention includes methods of treatment wherein a patient who is undergoing, or has recently undergone, standard lipid modifying therapy (e.g. a statin) is administered an ANGPTL8 inhibitor plus an ANGPTL3 inhibitor according to a particular dosing amount and frequency, and wherein the ANGPTL8 inhibitor and the ANGPTL3 inhibitor are administered as an add-on to the patient's pre-existing lipid modifying therapy (if applicable), such as an add-on to the patient's pre-existing daily therapeutic statin regimen.

For example, the methods of the present invention include add-on therapeutic regimens wherein the ANGPTL8 inhibitor and the ANGPTL3 inhibitor are administered as add-on therapy to the same stable daily therapeutic statin regimen (i.e., same dosing amount of statin) that the patient was on prior to receiving the ANGPTL8 and ANGPTL3 inhibitors. In other embodiments, the ANGPTL8 and ANGPTL3 inhibitors are administered as add-on therapy to a therapeutic statin regimen comprising a statin in an amount that is more than or less than the dose of statin the patient was on prior to receiving the ANGPTL8 and ANGPTL3 inhibitors. For example, after starting a therapeutic regimen comprising a ANGPTL8 inhibitor and an ANGPTL3 inhibitor administered at particular dosing frequencies and amounts, the daily dose of statin administered or prescribed to the patient may (a) stay the same, (b) increase, or (c) decrease (e.g., up-titrate or down-titrate) in comparison to the daily statin dose the patient was taking before starting the ANGPTL8 and ANGPTL3 inhibitors therapeutic regimen, depending on the therapeutic needs of the patient.

### Therapeutic Efficacy

The methods of the present invention may result in the reduction in serum levels of one or more lipid components selected from the group consisting of total cholesterol, LDL-C, non-HDL-C, ApoB100, VLDL-C, triglyceride (TG), Lp(a) and remnant cholesterol. For example, according to certain embodiments of the present invention, administration of an ANGPTL8 inhibitor in combination with an ANGPTL3 inhibitor to a suitable subject will result in a mean percent reduction from baseline in serum low density lipoprotein cholesterol (LDL-C) of at least about 25%, 30%, 40%, 50%, 60%, or greater; a mean percent reduction from baseline in ApoB100 of at least about 25%, 30%, 40%, 50%, 60%, or greater; a mean percent reduction from baseline in non-HDL-C of at least about 25%, 30%, 40%, 50%, 60%, or greater; a mean percent reduction from baseline in total cholesterol of at least about 10%, 15%, 20%, 25%, 30%, 35%, or greater; a mean percent reduction from baseline in VLDL-C of at least about 5%, 10%, 15%, 20%, 25%, 30%, or greater; a mean percent reduction from baseline in triglycerides of at least about 5%, 10%, 15%, 20%, 25%, 30%, 35% or greater; and/or a mean percent reduction from baseline in Lp(a) of at least about 5%, 10%, 15%, 20%, 25%, or greater.

### ANGPTL8 Inhibitors and ANGPTL3 Inhibitors

The methods of the present invention comprise administering to a patient a therapeutic composition comprising an ANGPTL8 inhibitor and an ANGPTL3 inhibitor.

### ANGPTL8 Inhibitors

As used herein, a “ANGPTL8 inhibitor” is any agent, which binds to or interacts with human ANGPTL8 and inhibits the normal biological function of ANGPTL8 in vitro or in vivo. Non-limiting examples of categories of ANGPTL8 inhibitors include small molecule ANGPTL8 antagonists, nucleic acid-based inhibitors of ANGPTL8 expression or activity (e.g., siRNA or antisense), peptide-based molecules that specifically interact with ANGPTL8 (e.g., peptibodies), receptor molecules that specifically interact with ANGPTL8, proteins comprising a ligand-binding portion of an LDL receptor, ANGPTL8-binding scaffold molecules (e.g., DARPins, HEAT repeat proteins, ARM repeat proteins, tetratricopeptide repeat proteins, fibronectin-based scaffold constructs, and other scaffolds based on naturally occurring repeat proteins, etc., [see, e.g., Boersma and Pluckthun, 2011, Curr. Opin. Biotechnol. 22:849-857, and references cited therein]), and anti-ANGPTL8 aptamers or portions thereof. According to certain embodiments, ANGPTL8 inhibitors that can be used in the context of the present invention are anti-ANGPTL8 antibodies or antigen-binding fragments of antibodies that specifically bind human ANGPTL8.

The term “human angiopoietin-like protein 8” or “human ANGPTL8” or “hANGPTL8”, or “ANGPTL8”, is also referred to as “lipasin”, “RIFL”, or “betatrophin”. Human ANGPTL8 also refers to ANGPTL8 having the protein sequence shown in amino acids 1-177 of SEQ ID NO: 9, or a biologically active fragment thereof, as well as that shown in GenBank accession number NP_061157.3 (SEQ ID NO:19)). The activity of ANGPTL8 that can be neutralized, inhibited, blocked, abrogated, reduced or interfered with, by an antibody or antigen-binding fragment thereof of the invention, includes, but is not limited to, inhibition of LPL activity, or lowering of triglyceride levels in vivo and the like.

### ANGPTL3 Inhibitors

As used herein, an “ANGPTL3 inhibitor” is any agent, which binds to or interacts with human ANGPTL3 and inhibits the normal biological function of ANGPTL3 in vitro or in vivo. Non-limiting examples of categories of ANGPTL3 inhibitors include small molecule ANGPTL3 antagonists, nucleic acid-based inhibitors of ANGPTL3 expression or activity (e.g., siRNA or antisense), peptide-based molecules that specifically interact with ANGPTL3 (e.g., peptibodies), receptor molecules that specifically interact with ANGPTL3, ANGPTL3-binding scaffold molecules (e.g., DARPins, HEAT repeat proteins, ARM repeat proteins, tetratricopeptide repeat proteins, fibronectin-based scaffold constructs, and other scaffolds based on naturally occurring repeat proteins, etc., [see, e.g., Boersma and Pluckthun, 2011, Curr. Opin. Biotechnol. 22:849-857, and references cited therein]), and anti-ANGPTL3 aptamers or portions thereof. According to certain embodiments, ANGPTL3 inhibitors that can be used in the context of the present invention are anti-ANGPTL3 antibodies or antigen-binding fragments of antibodies that specifically bind human ANGPTL3.

The term “human angiopoietin-like protein-3” or “human ANGPTL3” or “hANGPTL3”, as used herein, refers to ANGPTL3 having the amino acid sequence of SEQ ID NO: 18 (see also NCBI Accession NP_055310), or a biologically active fragment thereof.

The term “antibody”, as used herein, is intended to refer to immunoglobulin molecules comprising four polypeptide chains, two heavy (H) chains and two light (L) chains inter-connected by disulfide bonds, as well as multimers thereof (e.g., lgM). Each heavy chain comprises a heavy chain variable region (abbreviated herein as HCVR or V_{H}) and a heavy chain constant region. The heavy chain constant region comprises three domains, CH_{H}1, CH_{H}2 and CH_3. Each light chain comprises a light chain variable region (abbreviated herein as LCVR or V_{L}) and a light chain constant region. The light chain constant region comprises one domain (C_{L}1). The V_{H} and V_{L} regions can be further subdivided into regions of hypervariability, termed complementary determining regions (CDRs), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. In different embodiments of the invention, the FRs of the anti-ANGPTL 8 antibody (or antigen-binding portion thereof) may be identical to the human germline sequences, or may be naturally or artificially modified. An amino acid consensus sequence may be defined based on a side-by-side analysis of two or more CDRs.

The term “antibody,” as used herein, also includes antigen-binding fragments of full antibody molecules. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of antigen-binding fragments include: (i) Fab fragments; (ii) F(ab')2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementary determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g. monovalent nanobodies, bivalent nanobodies, etc.), small modular immunopharmaceuticals (SMIPs), and shark variable IgNAR domains, are also encompassed within the expression “antigen-binding fragment,” as used herein.

An antigen-binding fragment of an antibody will typically comprise at least one variable domain. The variable domain may be of any size or amino acid composition and will generally comprise at least one CDR, which is adjacent to or in frame with one or more framework sequences. In antigen-binding fragments having a V_{H} domain associated with a V_{L} domain, the V_{H} and V_{L} domains may be situated relative to one another in any suitable arrangement. For example, the variable region may be dimeric and contain V_{H}-V_{H}, V_{H}-V_{L} or V_{L}-V_{L} dimers. Alternatively, the antigen-binding fragment of an antibody may contain a monomeric V_{H} or V_{L} domain.

In certain embodiments, an antigen-binding fragment of an antibody may contain at least one variable domain covalently linked to at least one constant domain. Non-limiting, exemplary configurations of variable and constant domains that may be found within an antigen-binding fragment of an antibody of the present invention include: (i) V_{H}-C_{H}1; (ii) V_{H}-C_{L}2; (iii) V_{H}-C_{H}3; (iv) V_{H}-C_{H}1-C_{H}2: (v) V_{H}-C_{H}1-C_{H}2-C_{H}3; (vi) V_{H}-C_{H}2-C_{H}3; (vii) V_{H}-C_{L}; (viii) V_{L}-C_{H}1; (ix) V_{L}-C_{H}2; (x) V_{L}-C_{H}3; (xi) V_{L}-C_{H}1-C_{H}2; (xii) V_{L}-C_{H}1-C_{H}2-C_{H}3; (xiii) V_{L}-C_{H}2-C_{H}3; and (xiv) V_{L}-C_{L}. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60 or more) amino acids, which result in a flexible or semi-flexible linkage between adjacent variable and/or constant domains in a single polypeptide molecule. Moreover, an antigen-binding fragment of an antibody of the present invention may comprise a homo-dimer or hetero-dimer (or other multimer) of any of the variable and constant domain configurations listed above in non-covalent association with one another and/or with one or more monomeric V_{H} or V_{L} domain (e.g., by disulfide bond(s)).

As with full antibody molecules, antigen-binding fragments may be monospecific or multi specific (e.g., bispecific). A multi specific antigen-binding fragment of an antibody will typically comprise at least two different variable domains, wherein each variable domain is capable of specifically binding to a separate antigen or to a different epitope on the same antigen. Any multi specific antibody format, including the exemplary bispecific antibody formats disclosed herein, may be adapted for use in the context of an antigen-binding fragment of an antibody of the present invention using routine techniques available in the art.

The constant region of an antibody is important in the ability of an antibody to fix complement and mediate cell-dependent cytotoxicity. Thus, the isotype of an antibody may be selected on the basis of whether it is desirable for the antibody to mediate cytotoxicity.

The term “human antibody”, as used herein, is intended to include antibodies having variable and constant regions derived from human germline immunoglobulin sequences. The human antibodies of the invention may nonetheless include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody”, as used herein, is not intended to include antibodies in which CDR sequences derived from the germline of another mammalian species, such as a mouse, have been grafted onto human framework sequences. The term includes antibodies recombinantly produced in a non-human mammal, or in cells of a non-human mammal. The term is not intended to include antibodies isolated from or generated in a human subject.

The term “recombinant human antibody”, as used herein, is intended to include all human antibodies that are prepared, expressed, created or isolated by recombinant means, such as antibodies expressed using a recombinant expression vector transfected into a host cell (described further below), antibodies isolated from a recombinant, combinatorial human antibody library (described further below), antibodies isolated from an animal (e.g., a mouse) that is transgenic for human immunoglobulin genes (see e.g., Taylor et al. (1992) Nucl. Acids Res. 20:6287-6295) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

Human antibodies can exist in two forms that are associated with hinge heterogeneity. In one form, an immunoglobulin molecule comprises a stable four-chain construct of approximately 150-160 kDa in which the dimers are held together by an inter-chain heavy chain disulfide bond. In a second form, the dimers are not linked via inter-chain disulfide bonds and a molecule of about 75-80 kDa is formed composed of a covalently coupled light and heavy chain (half-antibody). These forms have been extremely difficult to separate, even after affinity purification.

The frequency of appearance of the second form in various intact IgG isotypes is due to, but not limited to, structural differences associated with the hinge region isotype of the antibody. A single amino acid substitution in the hinge region of the human IgG4 hinge can significantly reduce the appearance of the second form (Angal et al. (1993) Molecular Immunology 30:105) to levels typically observed using a human IgG1 hinge. The instant invention encompasses antibodies having one or more mutations in the hinge, C_{H}2 or C_{H}3 region which may be desirable, for example, in production, to improve the yield of the desired antibody form.

An “isolated antibody,” as used herein, means an antibody that has been identified and separated and/or recovered from at least one component of its natural environment. For example, an antibody that has been separated or removed from at least one component of an organism, or from a tissue or cell in which the antibody naturally exists or is naturally produced, is an “isolated antibody” for purposes of the present invention. An isolated antibody also includes an antibody in situ within a recombinant cell. Isolated antibodies are antibodies that have been subjected to at least one purification or isolation step. According to certain embodiments, an isolated antibody may be substantially free of other cellular material and/or chemicals.

The term “specifically binds,” or the like, means that an antibody or antigen-binding fragment thereof forms a complex with an antigen that is relatively stable under physiologic conditions. Methods for determining whether an antibody specifically binds to an antigen are well known in the art and include, for example, equilibrium dialysis, surface plasmon resonance, and the like. For example, an antibody that “specifically binds” ANGPTL8, or that “specifically binds” ANGPTL3, as used in the context of the present invention, includes antibodies that bind ANGPTL8, or ANGPTL3, or a portion thereof with a KD of less than about 1000 nM, less than about 500 nM, less than about 300 nM, less than about 200 nM, less than about 100 nM, less than about 90 nM, less than about 80 nM, less than about 70 nM, less than about 60 nM, less than about 50 nM, less than about 40 nM, less than about 30 nM, less than about 20 nM, less than about 10 nM, less than about 5 nM, less than about 4 nM, less than about 3 nM, less than about 2 nM, less than about 1 nM or less than about 0.5 nM, as measured in a surface plasmon resonance assay. An isolated antibody that specifically binds human ANGPTL8, or human ANGPTL3, however, has cross-reactivity to other antigens, such as ANGPTL8 molecules, or ANGPTL3 molecules from other (non-human) species.

The anti-ANGPTL8 and the anti-ANGPTL3 antibodies useful for the methods of the present invention may comprise one or more amino acid substitutions, insertions and/or deletions in the framework and/or CDR regions of the heavy and light chain variable domains as compared to the corresponding germline sequences from which the antibodies were derived. Such mutations can be readily ascertained by comparing the amino acid sequences disclosed herein to germline sequences available from, for example, public antibody sequence databases. The present invention includes methods involving the use of antibodies, and antigen-binding fragments thereof, which are derived from any of the amino acid sequences disclosed herein, wherein one or more amino acids within one or more framework and/or CDR regions are mutated to the corresponding residue(s) of the germline sequence from which the antibody was derived, or to the corresponding residue(s) of another human germline sequence, or to a conservative amino acid substitution of the corresponding germline residue(s) (such sequence changes are referred to herein collectively as “germline mutations”). A person of ordinary skill in the art, starting with the heavy and light chain variable region sequences disclosed herein, can easily produce numerous antibodies and antigen-binding fragments which comprise one or more individual germline mutations or combinations thereof. In certain embodiments, all of the framework and/or CDR residues within the V_{H} and/or V_{L} domains are mutated back to the residues found in the original germline sequence from which the antibody was derived. In other embodiments, only certain residues are mutated back to the original germline sequence, e.g., only the mutated residues found within the first 8 amino acids of FR1 or within the last 8 amino acids of FR4, or only the mutated residues found within CDR1, CDR2 or CDR3. In other embodiments, one or more of the framework and/or CDR residue(s) are mutated to the corresponding residue(s) of a different germline sequence (i.e., a germline sequence that is different from the germline sequence from which the antibody was originally derived). Furthermore, the antibodies of the present invention may contain any combination of two or more germline mutations within the framework and/or CDR regions, e.g., wherein certain individual residues are mutated to the corresponding residue of a particular germline sequence while certain other residues that differ from the original germline sequence are maintained or are mutated to the corresponding residue of a different germline sequence. Once obtained, antibodies and antigen-binding fragments that contain one or more germline mutations can be easily tested for one or more desired property such as, improved binding specificity, increased binding affinity, improved or enhanced antagonistic or agonistic biological properties (as the case may be), reduced immunogenicity, etc. The use of antibodies and antigen-binding fragments obtained in this general manner are encompassed within the present invention.

The present invention also includes methods involving the use of anti-ANGPTL8, and anti-ANGPTL3 antibodies comprising variants of any of the HCVR, LCVR, and/or CDR amino acid sequences disclosed herein having one or more conservative substitutions. For example, the present invention includes the use of anti-ANGPTL8, and anti-ANGPTL3 antibodies having HCVR, LCVR, and/or CDR amino acid sequences with, e.g., 10 or fewer, 8 or fewer, 6 or fewer, 4 or fewer, etc. conservative amino acid substitutions relative to any of the HCVR, LCVR, and/or CDR amino acid sequences disclosed herein.

The term “surface plasmon resonance”, as used herein, refers to an optical phenomenon that allows for the analysis of real-time interactions by detection of alterations in protein concentrations within a biosensor matrix, for example using the BIAcore™ system (Biacore Life Sciences division of GE Healthcare, Piscataway, N.J.).

The term “K_{D}”, as used herein, is intended to refer to the equilibrium dissociation constant of a particular antibody-antigen interaction.

The term “epitope” refers to an antigenic determinant that interacts with a specific antigen binding site in the variable region of an antibody molecule known as a paratope. A single antigen may have more than one epitope. Thus, different antibodies may bind to different areas on an antigen and may have different biological effects. Epitopes may be either conformational or linear. A conformational epitope is produced by spatially juxtaposed amino acids from different segments of the linear polypeptide chain. A linear epitope is one produced by adjacent amino acid residues in a polypeptide chain. In certain circumstance, an epitope may include moieties of saccharides, phosphoryl groups, or sulfonyl groups on the antigen.

According to certain embodiments, the anti-ANGPTL8 and anti-ANGPTL3 antibodies used in the methods of the present invention are antibodies with pH-dependent binding characteristics. As used herein, the expression “pH-dependent binding” means that the antibody or antigen-binding fragment thereof exhibits “reduced binding to ANGPTL8 at acidic pH as compared to neutral pH” (for purposes of the present disclosure, both expressions may be used interchangeably), or that the antibody or antigen-binding fragment thereof exhibits “reduced binding to ANGPTL3 at acidic pH as compared to neutral pH” (for purposes of the present disclosure, both expressions may be used interchangeably). For the example, antibodies “with pH-dependent binding characteristics” includes antibodies and antigen-binding fragments thereof that bind either to ANGPTL8, or to ANGPTL3 with higher affinity at neutral pH than at acidic pH. In certain embodiments, the antibodies and antigen-binding fragments of the present invention bind ANGPTL8, or ANGPTL3 with at least 3, 5, 10, 15, 20, 25, 30, 35, 40, 45, 50, 55, 60, 65, 70, 75, 80, 85, 90, 95, 100, or more times higher affinity at neutral pH than at acidic pH.

According to this aspect of the invention, the anti-ANGPTL8 antibodies, or the ANGPTL3 antibodies with pH-dependent binding characteristics may possess one or more amino acid variations relative to the parental anti-ANGPTL8 antibody, or the parental anti-ANGPTL3 antibody. For example, an anti-ANGPTL8 antibody, or an anti-ANGPTL 3 antibody with pH-dependent binding characteristics may contain one or more histidine substitutions or insertions, e.g., in one or more CDRs of a parental anti-ANGPTL 8, or a parental anti-ANGPTL3 antibody. Thus, according to certain embodiments of the present invention, methods are provided comprising administering an anti-ANGPTL8 antibody and an anti-ANGPTL3 antibody which comprises CDR amino acid sequences (e.g., heavy and light chain CDRs) which are identical to the CDR amino acid sequences of a parental anti-ANGPTL8 antibody, or parental ANGPTL3 antibody except for the substitution of one or more amino acids of one or more CDRs of the parental antibody with a histidine residue. The anti-ANGPTL8 antibodies, or anti-ANGPTL 3 antibodies with pH-dependent binding may possess, e.g., 1, 2, 3, 4, 5, 6, 7, 8, 9, or more histidine substitutions, either within a single CDR of a parental antibody or distributed throughout multiple (e.g., 2, 3, 4, 5, or 6) CDRs of a parental anti-ANGPTL8 antibody, or a parental anti-ANGPTL3 antibody. For example, the present invention includes the use of anti-ANGPTL8 antibodies and anti-ANGPTL3 antibodies with pH-dependent binding comprising one or more histidine substitutions in HCDR1, one or more histidine substitutions in HCDR2, one or more histidine substitutions in HCDR3, one or more histidine substitutions in LCDR1, one or more histidine substitutions in LCDR2, and/or one or more histidine substitutions in LCDR3, of a parental anti-ANGPTL 8 antibody, or a parental anti-ANGPTL3 antibody.

As used herein, the expression “acidic pH” means a pH of 6.0 or less (e.g., less than about 6.0, less than about 5.5, less than about 5.0, etc.). The expression “acidic pH” includes pH values of about 6.0, 5.95, 5.90, 5.85, 5.8, 5.75, 5.7, 5.65, 5.6, 5.55, 5.5, 5.45, 5.4, 5.35, 5.3, 5.25, 5.2, 5.15, 5.1, 5.05, 5.0, or less. As used herein, the expression “neutral pH” means a pH of about 7.0 to about 7.4. The expression “neutral pH” includes pH values of about 7.0, 7.05, 7.1, 7.15, 7.2, 7.25, 7.3, 7.35, and 7.4.

A non-limiting example of an anti-ANGPTL8 antibody that can be used in the context of the present invention includes an anti-ANGPTL8 antibody comprising the three heavy chain complementary determining regions (HCDRs) contained in the heavy chain variable region (HCVR) of SEQ ID NO: 1 and the three light chain complementary determining regions (LCDRs) contained in the light chain variable region (LCVR) of SEQ ID NO: 5. In one embodiment, an anti-ANGPTL8 antibody that can be used in the context of the present invention includes an anti-ANGPTL8 antibody comprising the HCDR1 of SEQ ID NO: 2, the HCDR2 of SEQ ID NO: 3, the HCDR3 of SEQ ID NO: 4, the LCDR1 of SEQ ID NO: 6, the LCDR2 of SEQ ID NO: 7, and the LCDR3 of SEQ ID NO: 8. In one embodiment, the anti-ANGPTL8 antibody that can be used in the context of the present invention includes an anti-ANGPTL8 antibody comprising the HCVR/LCVR amino acid sequence pair of SEQ ID NOs: 1/5.

A non-limiting example of an anti-ANGPTL3 antibody that can be used in the context of the present invention includes evinacumab, which comprises the three heavy chain complementary determining regions (HCDRs) contained in the heavy chain variable region (HCVR) of SEQ ID NO: 10 and the three light chain complementary determining regions (LCDRs) contained in the light chain variable region (LCVR) of SEQ ID NO: 14. Evinacumab comprises the HCDR1 of SEQ ID NO: 11, the HCDR2 of SEQ ID NO: 12, the HCDR3 of SEQ ID NO: 13, the LCDR1 of SEQ ID NO: 15, the LCDR2 of SEQ ID NO: 16, and the LCDR3 of SEQ ID NO: 17. Evinacumab comprises the HCVR/LCVR amino acid sequence pair of SEQ ID NOs: 10/14.

### Preparation of Human Antibodies

Anti-ANGPTL8 antibodies and anti-ANGPTL3 antibodies can be made according to any method of antibody production/isolation known in the art. For example, antibodies for use in the methods of the present invention may be made by hybridoma technologies, by phage display, by yeast display, etc. Antibodies for use in the methods of the present invention may be, e.g., chimeric antibodies, humanized antibodies, or fully human antibodies.

Methods for generating human antibodies in transgenic mice are known in the art. Any such known methods can be used in the context of the present invention to make human antibodies that specifically bind ANGPTL8, or ANGPTL3.

For example, using VELOCIMMUNE™ technology (see, for example, U.S. Pat No. 6,596,541, Regeneron Pharmaceuticals) or any other known method for generating monoclonal antibodies, high affinity chimeric antibodies to ANGPTL8, or to ANGPTL3 are initially isolated having a human variable region and a mouse constant region. The VELOCIMMUNE® technology involves generation of a transgenic mouse having a genome comprising human heavy and light chain variable regions operably linked to endogenous mouse constant region loci such that the mouse produces an antibody comprising a human variable region and a mouse constant region in response to antigenic stimulation. The DNA encoding the variable regions of the heavy and light chains of the antibody are isolated and operably linked to DNA encoding the human heavy and light chain constant regions. The DNA is then expressed in a cell capable of expressing the fully human antibody.

Generally, a VELOCIMMUNE® mouse is challenged with the antigen of interest, and lymphatic cells (such as B-cells) are recovered from the mice that express antibodies. The lymphatic cells may be fused with a myeloma cell line to prepare immortal hybridoma cell lines, and such hybridoma cell lines are screened and selected to identify hybridoma cell lines that produce antibodies specific to the antigen of interest. DNA encoding the variable regions of the heavy chain and light chain may be isolated and linked to desirable isotypic constant regions of the heavy chain and light chain. Such an antibody protein may be produced in a cell, such as a CHO cell. Alternatively, DNA encoding the antigen-specific chimeric antibodies or the variable domains of the light and heavy chains may be isolated directly from antigen-specific lymphocytes.

Initially, high affinity chimeric antibodies are isolated having a human variable region and a mouse constant region. The antibodies are characterized and selected for desirable characteristics, including affinity, selectivity, epitope, etc., using standard procedures known to those skilled in the art. The mouse constant regions are replaced with a desired human constant region to generate the fully human antibody of the invention, for example wild-type or modified IgG1 or IgG4. While the constant region selected may vary according to specific use, high affinity antigen-binding and target specificity characteristics reside in the variable region.

In general, the antibodies that can be used in the methods of the present invention possess high affinities, as described above, when measured by binding to antigen either immobilized on solid phase or in solution phase. The mouse constant regions are replaced with desired human constant regions to generate the fully human antibodies of the invention. While the constant region selected may vary according to specific use, high affinity antigen-binding and target specificity characteristics reside in the variable region.

Specific examples of human antibodies or antigen-binding fragments of antibodies that specifically bind ANGPTL8, which can be used in the context of the methods of the present invention include antibodies or antigen-binding proteins comprising the six CDRs (HCDR1, HCDR2, HCDR3, LCDR1, LCDR2 and LCDR3) from the heavy and light chain variable region (HCVR/LCVR) amino acid sequence pair comprising SEQ ID NOs: 1/5.

In certain embodiments of the present invention, the anti-ANGPTL8 antibody, or antigen-binding fragment thereof, that can be used in the methods of the present invention comprises heavy and light chain complementary determining regions (HCDR1-HCDR2-HCDR3/LCDR1-LCDR2-LCDR3) comprising the amino acid sequences of SEQ ID NOs: 2, 3, 4, 6, 7 and 8.

In certain embodiments of the present invention, the anti-ANGPTL8 antibody, or antigen-binding fragment thereof, that can be used in the methods of the present invention comprises an HCVR having the amino acid sequence of SEQ ID NO:1 and an LCVR having the amino acid sequence of SEQ ID NO:5.

Specific examples of human antibodies or antigen-binding fragments of antibodies that specifically bind ANGPTL3, which can be used in the context of the methods of the present invention include antibodies or antigen-binding proteins comprising the six CDRs (HCDR1, HCDR2, HCDR3, LCDR1, LCDR2 and LCDR3) from the heavy and light chain variable region (HCVR/LCVR) amino acid sequence pair comprising SEQ ID NOs: 10/14.

In certain embodiments of the present invention, the anti-ANGPTL3 antibody, or antigen-binding fragment thereof, that can be used in the methods of the present invention comprises heavy and light chain complementary determining regions (HCDR1-HCDR2-HCDR3/LCDR1-LCDR2-LCDR3) comprising the amino acid sequences of SEQ ID NOs:11, 12, 13, 15, 16 and 17.

In certain embodiments of the present invention, the anti-ANGPTL3 antibody, or antigen-binding fragment thereof, that can be used in the methods of the present invention comprises an HCVR having the amino acid sequence of SEQ ID NO:10 and an LCVR having the amino acid sequence of SEQ ID NO:14.

### Pharmaceutical Compositions and Methods of Administration

The present invention includes methods, which comprise administering an ANGPTL8 inhibitor to a patient in combination with an ANGPTL3 inhibitor, wherein the ANGPTL8 inhibitor and the ANGPTL3 inhibitor are contained within the same, or in different pharmaceutical compositions. The pharmaceutical compositions of the invention are formulated with suitable carriers, excipients, and other agents that provide suitable transfer, delivery, tolerance, and the like. A multitude of appropriate formulations can be found in the formulary known to all pharmaceutical chemists: Remington's Pharmaceutical Sciences, Mack Publishing Company, Easton, PA. These formulations include, for example, powders, pastes, ointments, jellies, waxes, oils, lipids, lipid (cationic or anionic) containing vesicles (such as LIPOFECTIN™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. See also Powell et al. “Compendium of excipients for parenteral formulations” PDA (1998) J Pharm Sci Technol 52:238-311.

Exemplary pharmaceutical formulations comprising anti-ANGPTL8 antibodies, and/or ANGPTL3 antibodies that can be used in the context of the present invention include any of the formulations as set forth in U.S. Pat. No. 8,795,669, or in WO2013/166448, or WO2012/168491.

Various delivery systems are known and can be used to administer the pharmaceutical composition of the invention, e.g., encapsulation in liposomes, microparticles, microcapsules, recombinant cells capable of expressing the mutant viruses, receptor mediated endocytosis (see, e.g., Wu et al., 1987, J. Biol. Chem. 262:4429-4432). Methods of administration include, but are not limited to, intradermal, intramuscular, intraperitoneal, intravenous, subcutaneous, intranasal, epidural, and oral routes. The composition may be administered by any convenient route, for example by infusion or bolus injection, by absorption through epithelial or mucocutaneous linings (e.g., oral mucosa, rectal and intestinal mucosa, etc.) and may be administered together with other biologically active agents.

A pharmaceutical composition of the present invention can be delivered subcutaneously or intravenously with a standard needle and syringe. In addition, with respect to subcutaneous delivery, a pen delivery device readily has applications in delivering a pharmaceutical composition of the present invention. Such a pen delivery device can be reusable or disposable. A reusable pen delivery device generally utilizes a replaceable cartridge that contains a pharmaceutical composition. Once all of the pharmaceutical composition within the cartridge has been administered and the cartridge is empty, the empty cartridge can readily be discarded and replaced with a new cartridge that contains the pharmaceutical composition. The pen delivery device can then be reused. In a disposable pen delivery device, there is no replaceable cartridge. Rather, the disposable pen delivery device comes prefilled with the pharmaceutical composition held in a reservoir within the device. Once the reservoir is emptied of the pharmaceutical composition, the entire device is discarded.

Numerous reusable pen and auto injector delivery devices have applications in the subcutaneous delivery of a pharmaceutical composition of the present invention. Examples include, but are not limited to AUTOPEN™ (Owen Mumford, Inc., Woodstock, UK), DISETRONIC™ pen (Disetronic Medical Systems, Bergdorf, Switzerland), HUMALOG MIX 75/25198 pen, HUMALOG™ pen, HUMALIN 70/30™ pen (Eli Lilly and Co., Indianapolis, Ind.), NOVOPEN™ I, II and III (Novo Nordisk, Copenhagen, Denmark), NOVOPEN JUNIOR™ (Novo Nordisk, Copenhagen, Denmark), BD™ pen (Becton Dickinson, Franklin Lakes, N.J.), OPTIPEN™, OPTIPEN PRO™, OPTIPEN STARLET™, and OPTICLIK™ (Sanofi-Aventis, Frankfurt, Germany), to name only a few. Examples of disposable pen delivery devices having applications in subcutaneous delivery of a pharmaceutical composition of the present invention include, but are not limited to the SOLOSTAR™ pen (Sanofi-Aventis), the FLEXPEN™ (Novo Nordisk), and the KWIKPEN™ (Eli Lilly), the SURECLICK™ Auto injector (Amgen, Thousand Oaks, Calif.), the PENLET™ (Haselmeier, Stuttgart, Germany), the EPIPEN (Dey, L.P.), and the HUMIRA™ Pen (Abbott Labs, Abbott Park Ill.), to name only a few.

In certain situations, the pharmaceutical composition can be delivered in a controlled release system. In one embodiment, a pump may be used (see Langer, supra; Sefton, 1987, CRC Crit. Ref. Biomed. Eng. 14:201). In another embodiment, polymeric materials can be used; see, Medical Applications of Controlled Release, Langer and Wise (eds.), 1974, CRC Pres., Boca Raton, Fla. In yet another embodiment, a controlled release system can be placed in proximity of the composition's target, thus requiring only a fraction of the systemic dose (see, e.g., Goodson, 1984, in Medical Applications of Controlled Release, supra, vol. 2, pp. 115-138). Other controlled release systems are discussed in the review by Langer, 1990, Science 249:1527-1533.

The injectable preparations may include dosage forms for intravenous, subcutaneous, intracutaneous and intramuscular injections, drip infusions, etc. These injectable preparations may be prepared by known methods. For example, the injectable preparations may be prepared, e.g., by dissolving, suspending or emulsifying the antibody or its salt described above in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant [e.g., polysorbate 80, HCO-50 (polyoxyethylene (50 mol) adduct of hydrogenated castor oil)], etc. As the oily medium, there are employed, e.g., sesame oil, soybean oil, etc., which may be used in combination with a solubilizing agent such as benzyl benzoate, benzyl alcohol, etc. The injection thus prepared is preferably filled in an appropriate ampoule.

Advantageously, the pharmaceutical compositions for oral or parenteral use described above are prepared into dosage forms in a unit dose suited to fit a dose of the active ingredients. Such dosage forms in a unit dose include, for example, tablets, pills, capsules, injections (ampoules), suppositories, etc.

### Dosage

The amount of an ANGPTL8 inhibitor (e.g., anti-ANGPTL8 antibody), or an ANGPTL3 inhibitor (e.g., anti-ANGPTL3 antibody) administered to a subject according to the methods of the present invention is, generally, a therapeutically effective amount. As used herein, the phrase “therapeutically effective amount of a ANGPTL8 inhibitor” means a dose of a ANGPTL8 inhibitor, when administered in combination with an ANGPTL3 inhibitor, results in a detectable reduction (at least about 5%, 10%, 15%, 20%, 25%, 30%, 35%, 40%, 45%, 50%, 55%, 60%, 65%, 70%, 75%, or more from baseline) in one or more parameters selected from the group consisting of total cholesterol, LDL-C, ApoB100, non-HDL-C, VLDL-C, triglycerides, Lp(a) and remnant cholesterol, or an amount that reduces or eliminates a patient's need for other therapeutic interventions, such as, lipoprotein apheresis, or that reduces a patient's normalized rate of apheresis.

In the case of an anti-ANGPTL8 antibody, a therapeutically effective amount can be from about 0.05 mg to about 600 mg, e.g., about 0.05 mg, about 0.1 mg, about 1.0 mg, about 1.5 mg, about 2.0 mg, about 10 mg, about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, or about 600 mg, of the anti-ANGPTL8 antibody. According to certain exemplary embodiments of the present invention, a therapeutically effective amount of an anti-ANGPTL8 antibody is 75 mg, 150 mg or 300 mg (e.g., in the case of alirocumab), or 140 mg or 420 mg (e.g., in the case of evolocumab). Other dosing amounts of ANGPTL8 inhibitors will be apparent to persons of ordinary skill in the art and are contemplated within the scope of the present invention.

The amount of anti-ANGPTL8 antibody contained within the individual doses may be expressed in terms of milligrams of antibody per kilogram of patient body weight (i.e., mg/kg). For example, the anti-ANGPTL8 antibody may be administered to a patient at a dose of about 0.0001 to about 10 mg/kg of patient body weight.

The amount of ANGPTL3 inhibitor (e.g., anti-ANGPTL3 antibody) administered to a subject according to the methods of the present invention is, generally, a therapeutically effective amount. As used herein, the phrase “therapeutically effective amount of an ANGPTL3 inhibitor” means a dose of ANGPTL3 inhibitor, when combined with a ANGPTL8 inhibitor, results in a detectable reduction (at least about 5%, 10%, 15%, 20%, 25%, 30%, 35%, 40%, 45%, 50%, 55%, 60%, 65%, 70%, 75%, or more from baseline) in one or more parameters selected from the group consisting of total cholesterol, LDL-C, ApoB100, non-HDL-C, VLDL-C, triglycerides, Lp(a) and remnant cholesterol, or an amount that prevents or attenuates atherosclerosis in a subject (as described elsewhere herein).

In the case of an anti-ANGPTL3 antibody, a therapeutically effective amount can be from about 0.05 mg to about 600 mg, e.g., about 0.05 mg, about 0.1 mg, about 1.0 mg, about 1.5 mg, about 2.0 mg, about 10 mg, about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, or about 600 mg, of the anti-ANGPTL3 antibody. Other dosing amounts of ANGPTL3 inhibitors will be apparent to persons of ordinary skill in the art and are contemplated within the scope of the present invention.

The amount of anti-ANGPTL3 antibody contained within the individual doses may be expressed in terms of milligrams of antibody per kilogram of patient body weight (i.e., mg/kg). For example, the anti-ANGPTL3 antibody may be administered to a patient at a dose of about 0.0001 to about 10 mg/kg of patient body weight.

### Combination Therapies

As described elsewhere herein, the methods of the present invention may comprise administering an ANGPTL8 inhibitor in combination with an ANGPTL3 inhibitor to a patient who is non-responsive to, inadequately controlled by, or intolerant to a standard lipid lowering therapy. In certain embodiments, the need for further administration of the lipid lowering therapy may be eliminated altogether. In certain embodiments, the combined use of the ANGPTL8 inhibitor with the ANGPTL3 inhibitor may be used in combination with (“on top of”) the patient's previously prescribed lipid lowering therapy. For example, in the context of lowering at least one lipid/lipoprotein parameter in a patient suffering from hyperlipidaemia (e.g. hypercholesterolemia, or hypertriglyceridemia), wherein the patient is non-responsive to, inadequately controlled by, or intolerant to a standard lipid lowering therapy, a combination of a ANGPTL8 inhibitor with an ANGPTL3 inhibitor may be administered to a patient in combination with a stable daily therapeutic statin regimen. Exemplary daily therapeutic statin regimens that a ANGPTL8 inhibitor plus an ANGPTL3 inhibitor may be administered in combination with in the context of the present invention include, e.g., atorvastatin (10, 20, 40 or 80 mg daily), (atorvastatin/ezetimibe 10/10 or 40/10 mg daily), rosuvastatin (5, 10 or 20 mg daily), cerivastatin (0.4 or 0.8 mg daily), pitavastatin (1, 2 or 4 mg daily), fluvastatin (20, 40 or 80 mg daily), simvastatin (5, 10, 20, 40 or 80 mg daily), simvastatin/ezetimibe (10/10, 20/10, 40/10 or 80/10 mg daily), lovastatin (10, 20, 40 or 80 mg daily), pravastatin (10, 20, 40 or 80 mg daily), and combinations thereof. Other lipid modifying therapies that a ANGPTL8 inhibitor plus an ANGPTL3 inhibitor may be administered in combination with in the context of the present invention include, e.g., (1) an agent which inhibits cholesterol uptake and or bile acid re-absorption (e.g., ezetimibe); (2) an agent which increase lipoprotein catabolism (such as niacin); and/or (3) activators of the LXR transcription factor that plays a role in cholesterol elimination such as 22-hydroxycholesterol.

A non-limiting example of an anti-ANGPTL8 antibody that can be used in the context of the present invention includes an antibody comprising the HCVR/LCVR amino acid sequence pair of SEQ ID NOs: 1/5, or an antigen-binding portion thereof.

A non-limiting example of an ANGPTL3 antibody to be used in the context of the present invention includes evinacumab, comprising the HCVR/LCVR amino acid sequence pair of SEQ ID NOs: 10/14, or an antigen-binding portion thereof.

### Administration Regimens

According to certain embodiments of the present invention, multiple doses of an ANGPTL8 inhibitor (i.e., a pharmaceutical composition comprising an ANGPTL8 inhibitor) and an ANGPTL3 inhibitor (i.e., a pharmaceutical composition comprising an ANGPTL3 inhibitor) may be administered to a subject over a defined time course (e.g., on top of a daily therapeutic statin regimen or other background lipid modifying therapy). The methods according to this aspect of the invention comprise sequentially administering to a subject multiple doses of an ANGPTL8 inhibitor and an ANGPTL3 inhibitor. As used herein, “sequentially administering” means that each dose of ANGPTL8 inhibitor and ANGPTL3 inhibitor is administered to the subject at a different point in time, e.g., on different days separated by a predetermined interval (e.g., hours, days, weeks or months). The present invention includes methods which comprise sequentially administering to the patient a single initial dose of an ANGPTL8 inhibitor and an ANGPTL3 inhibitor, followed by one or more secondary doses of the ANGPTL8 inhibitor and ANGPTL3 inhibitor, and optionally followed by one or more tertiary doses of the ANGPTL8 inhibitor and ANGPTL3 inhibitor.

The terms “initial dose,” “secondary doses,” and “tertiary doses,” refer to the temporal sequence of administration of the individual doses of a pharmaceutical composition comprising an ANGPTL8 inhibitor and the ANGPTL3 inhibitor. Thus, the “initial dose” is the dose which is administered at the beginning of the treatment regimen (also referred to as the “baseline dose”); the “secondary doses” are the doses which are administered after the initial dose; and the “tertiary doses” are the doses which are administered after the secondary doses. The initial, secondary, and tertiary doses may all contain the same amount of the ANGPTL8 inhibitor and the ANGPTL3 inhibitor, but generally may differ from one another in terms of frequency of administration. In certain embodiments, however, the amount of ANGPTL8 inhibitor and the ANGPTL3 inhibitor contained in the initial, secondary and/or tertiary doses varies from one another (e.g., adjusted up or down as appropriate) during the course of treatment. In certain embodiments, two or more (e.g., 2, 3, 4, or 5) doses are administered at the beginning of the treatment regimen as “loading doses” followed by subsequent doses that are administered on a less frequent basis (e.g., “maintenance doses”).

According to exemplary embodiments of the present invention, each secondary and/or tertiary dose is administered 1 to 26 (e.g., 1, 1½, 2, 2½, 3, 3½, 4, 4½, 5, 5½, 6, 6½, 7, 7½, 8, 8½, 9, 9½, 10, 10½, 11, 11½, 12, 12½, 13, 13½, 14, 14½, 15, 15½, 16, 16½, 17, 17½, 18, 18½, 19, 19½, 20, 20½, 21, 21½, 22, 22½, 23, 23½, 24, 24½, 25 25½, 26, 26½ , or more) weeks after the immediately preceding dose. The phrase “the immediately preceding dose,” as used herein, means, in a sequence of multiple administrations, the dose of antigen-binding molecule, which is administered to a patient prior to the administration of the very next dose in the sequence with no intervening doses.

The methods according to this aspect of the invention may comprise administering to a patient any number of secondary and/or tertiary doses of an ANGPTL8 inhibitor and ANGPTL3 inhibitor. For example, in certain embodiments, only a single secondary dose is administered to the patient. In other embodiments, two or more (e.g., 2, 3, 4, 5, 6, 7, 8, or more) secondary doses are administered to the patient. Likewise, in certain embodiments, only a single tertiary dose is administered to the patient. In other embodiments, two or more (e.g., 2, 3, 4, 5, 6, 7, 8, or more) tertiary doses are administered to the patient.

In embodiments involving multiple secondary doses, each secondary dose may be administered at the same frequency as the other secondary doses. For example, each secondary dose may be administered to the patient 1 to 2, 4, 6, 8 or more weeks after the immediately preceding dose. Similarly, in embodiments involving multiple tertiary doses, each tertiary dose may be administered at the same frequency as the other tertiary doses. For example, each tertiary dose may be administered to the patient 1 to 2, 4, 6, 8 or more weeks after the immediately preceding dose. Alternatively, the frequency at which the secondary and/or tertiary doses are administered to a patient can vary over the course of the treatment regimen. The frequency of administration may also be adjusted during the course of treatment by a physician depending on the needs of the individual patient following clinical examination.

### EXAMPLES

The following examples are put forth so as to provide those of ordinary skill in the art with a complete disclosure and description of how to make and use the methods and compositions of the invention, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1

### Generation of Human Antibodies to Human ANGPTL8

Anti-ANGPTL8 antibodies were obtained by immunizing a VELOCIMMUNE® mouse (i.e., an engineered mouse comprising DNA encoding human immunoglobulin heavy and kappa light chain variable regions) with an immunogen comprising a recombinant human ANGPTL8 expressed with a C-terminal mouse IgG2a Fc tag (See SEQ ID NO: 9). The antibody immune response was monitored by an ANGPTL8-specific immunoassay. When a desired immune response was achieved, several fully human anti-ANGPTL8 antibodies were generated from antigen-positive B cells as described in U.S. 2007/0280945A1, incorporated by reference herein in its entirety.

The exemplary ANGPTL8 inhibitor used in the following Example is the human anti-ANGPTL8 antibody designated “H4H15341P”. A heavy chain variable region (HCVR) comprising SEQ ID NO:1 and a light chain variable domain (LCVR) comprising SEQ ID NO:5; a heavy chain complementary determining region 1 (HCDR1) comprising SEQ ID NO:2, a HCDR2 comprising SEQ ID NO:3, a HCDR3 comprising SEQ ID NO:4, a light chain complementary determining region 1 (LCDR1) comprising SEQ ID NO:6, a LCDR2 comprising SEQ ID NO:7 and a LCDR3 comprising SEQ ID NO:8.

### Example 2

### Generation of Human Antibodies to Human ANGPTL3

Human anti-ANGPTL3 antibodies were generated as described in U.S. Pat. No. 9,018,356. The exemplary ANGPTL3 inhibitor used in the following Example is the human anti-ANGPTL3 antibody designated “H4H1276S,” also known as “evinacumab.” H4H1276S has the following amino acid sequence characteristics: a heavy chain variable region (HCVR) comprising SEQ ID NO:10 and a light chain variable domain (LCVR) comprising SEQ ID NO:14; a heavy chain complementary determining region 1 (HCDR1) comprising SEQ ID NO:11, a HCDR2 comprising SEQ ID NO:12, a HCDR3 comprising SEQ ID NO:13, a light chain complementary determining region 1 (LCDR1) comprising SEQ ID NO:15, a LCDR2 comprising SEQ ID NO:16 and a LCDR3 comprising SEQ ID NO:17.

### Example 3

### In Vivo Effect of Anti-ANGPTL3 Antibody on Circulating Triglyceride and Cholesterol Levels in ANGPTL8 Knockout (KO) and Wild Type (WT) Mice

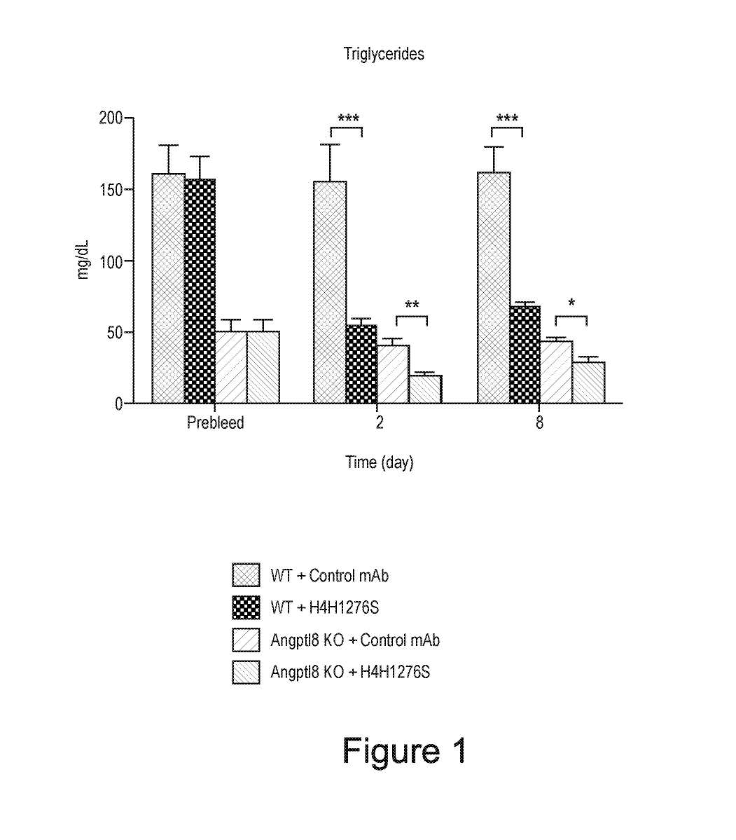

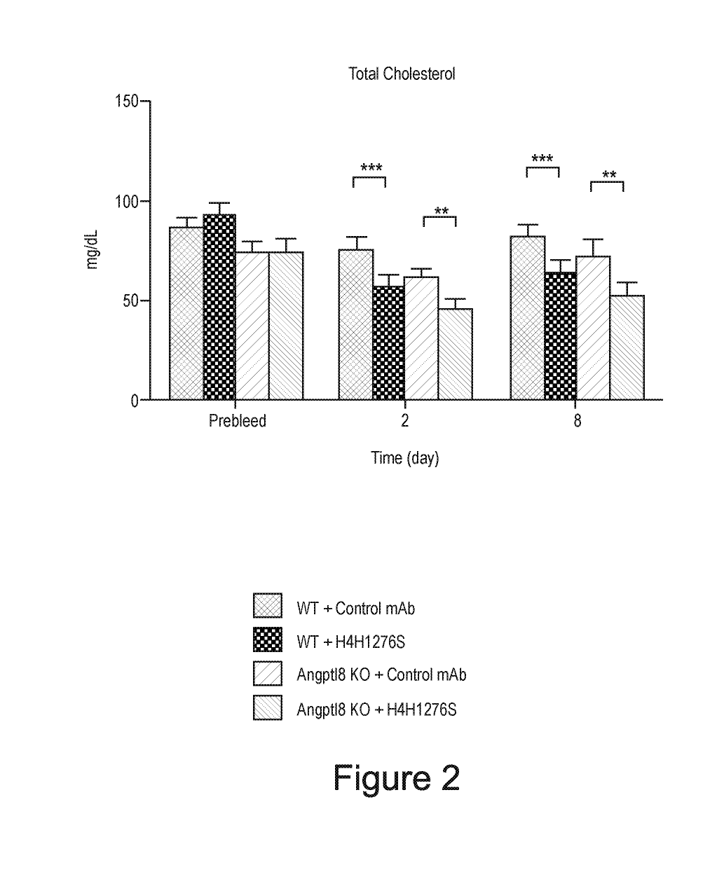

The effects of ANGPTL3 antibody H4H1276S on serum triglycerides (TG) and total cholesterol were evaluated in Angptl8 knockout and wild-type mice. Mice were pre-bled at fasted-refed conditions (refed for 6 hours after overnight fast) 5 days before the experiment. The mice were sorted into groups (5 mice each per antibody per genotype) based on their TG and total cholesterol baseline values. The antibodies, isotype-matched (hlgG4) control with irrelevant specificity and H4H1276S (anti-ANGPTL3), were administered by single-dose subcutaneous injection on Day 0 of the study at 10mg/kg. Mice were bled at days 2 and 8 at fasted-refed conditions and TG and total cholesterol levels were determined in the serum by ADVIA® 1800 Chemistry System (Siemens). Averages were calculated for each time point. Results, expressed as (mean±SEM) are shown in FIGS. 1 and 2. FIG. 1 shows the results of triglyceride levels in both ANGPTL8 KO mice and WT mice. FIG. 2 shows the results of total cholesterol levels in both ANGPTL8 KO mice and WT mice.

### Results Summary:

The effect of ANGPTL3 antibody H4H1276S on circulating TG and total cholesterol levels was evaluated in Angptl8 knockout and wild-type mice. H4H1276S treatment led to significant reduction in circulating TG and total cholesterol level in wild-type mice, consistent with that reported earlier (Gusarova et al, 2015, J. Lipid Res. (2015), Jul;56(7):1308-17). H4H1276S also caused significant reduction in TG levels in Angptl8 knockout mice, which already had reduced baseline TG level due to Angptl8 deletion. Total cholesterol was also significantly reduced by H4H1276S treatment of Angptl8 knockout mice. These data suggest inhibition of both ANGPTL3 and ANGPTL8 may have an additive effect on circulating TG levels.

### Example 4

### In Vivo Effect of Anti-ANGPTL8 and Anti-ANGPTL3 and Their Combination on Circulating Triglycerides (TG) and Total Cholesterol (TC) in Humanized ANGPTL8 Mice

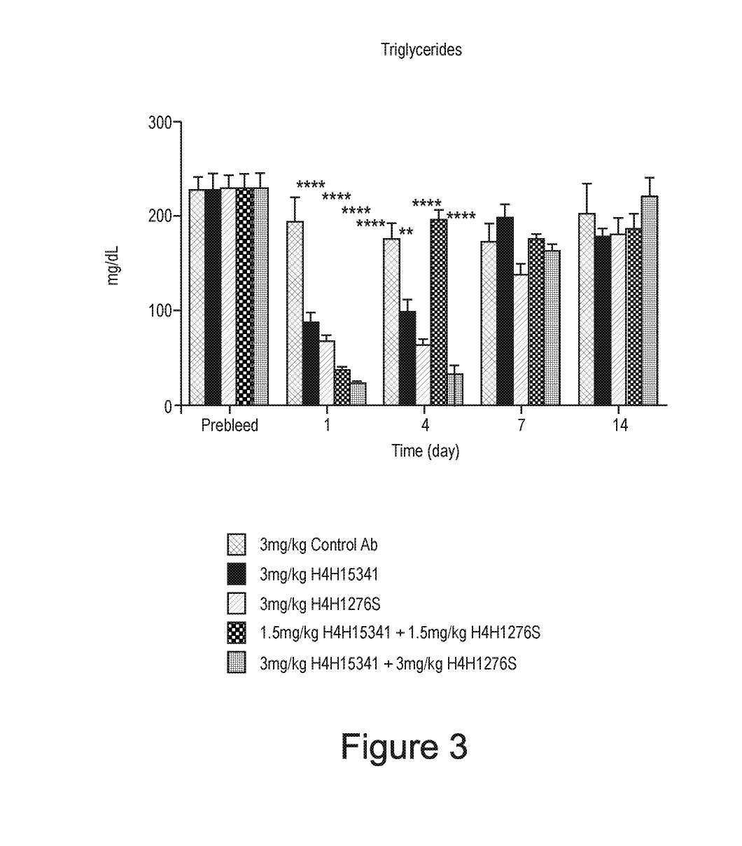

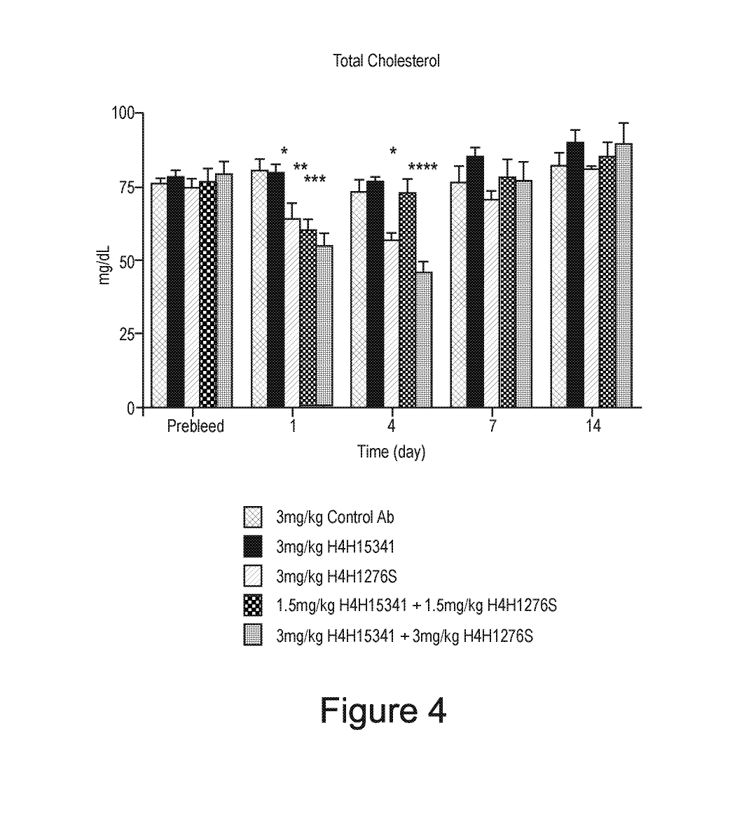

The effects of combinational treatment of anti-ANGPTL8 and anti-ANGPTL3 antibodies on serum triglycerides (TG) and total cholesterol levels were evaluated in humanized ANGPTL8 mice. Mice were pre-bled 6 days before the experiment at nonfasted conditions. The mice were sorted into groups (five mice each for each antibody or antibody combination tested) based on their baseline TG and body weights values. The antibodies were administered by single-dose subcutaneous injection on Day 0 of the study: isotype-matched (hlgG4) control with irrelevant specificity, H4H15341P and H4H1276S were administered at 3mg/kg dose and the combination of H4H15341P and H4H1276S were administered at 2 doses—1.5mg/kg or 3mg/kg of each. Mice were bled (nonfasted) at days 1, 4, 8 and 14 after the antibodies injection, TG and total cholesterol levels were determined in the serum by ADVIA® 1800 Chemistry System (Siemens). Averages were calculated for each time point. Results, expressed as (mean±SEM) are shown in FIGS. 3 and 4. FIG. 3 shows the effect on triglycerides when the ANGPTL3 or ANGPTL8 antibodies were used alone, or when they were used in combination. Measurements were made at days 1, 4, 7 and 14 after administration. FIG. 4 shows the effect on total cholesterol level when the ANGPTL3 or ANGPTL8 antibodies were used alone, or when they were used in combination. Measurements were made at days 1, 4, 7 and 14 after administration.

### Results Summary:

The effect of combinational treatment of H4H15341P (anti-hANGPTL8) and H4H1276S (anti-ANGPTL3) on circulating TG levels were tested in humanized ANGPTL8 mice. The administration of single mAb or combination led to significant reduction in circulating TG level where combinational treatment shown an additive effect on serum TG levels compared to the single mAb effects.

### Example 5

### Studies Regarding the Interaction of AngPTL3 and AngPTL8

Angiopoietin-like protein 3 (ANGPTL3) and ANGPTL8 are secreted proteins and inhibitors of lipoprotein lipase (LPL)—mediated plasma triglyceride clearance. How these ANGPTL proteins interact to regulate LPL activity to supply appropriate amounts of fatty acids to tissues for storage or oxidation was studied. ANGPTL3 inhibits LPL activity and increase serum triglycerides (TG) in mice independent of ANGPTL8. The effects on LPL activity and serum TG could be reversed with an ANGPTL3 blocking antibody. It was found that ANGPTL8 has a functional LPL inhibitory motif but requires ANGPTL3 to inhibit LPL and increase serum TG in mice. Site-directed mutagenesis revealed that the ability of ANGPTL8 to block LPL activity and increase serum TG did not require ANGPTL3 with a functional LPL inhibitory motif. An antibody to the C-terminus of ANGPTL8 (see Example 6) reversed LPL inhibition by ANGPTL8 in the presence of ANGPTL3. The antibody did not disrupt the ANGPTL8:ANGPTL3 complex but comes in close proximity to the LPL inhibitory motif in the N-terminus of ANGPTL8. Collectively, these data show that ANGPTL8 has a functional LPL inhibitory motif but can only inhibit LPL in the presence of ANGPTL3 (Haller, J. et al. Nature Scientific Reports, submitted 2017).

### Example 6

### Anti-ANGPTL8 Blocking Antibody Reverses ANGPTL8-Induced Inhibition of LPL Without Disrupting the ANGPTL3 Interaction

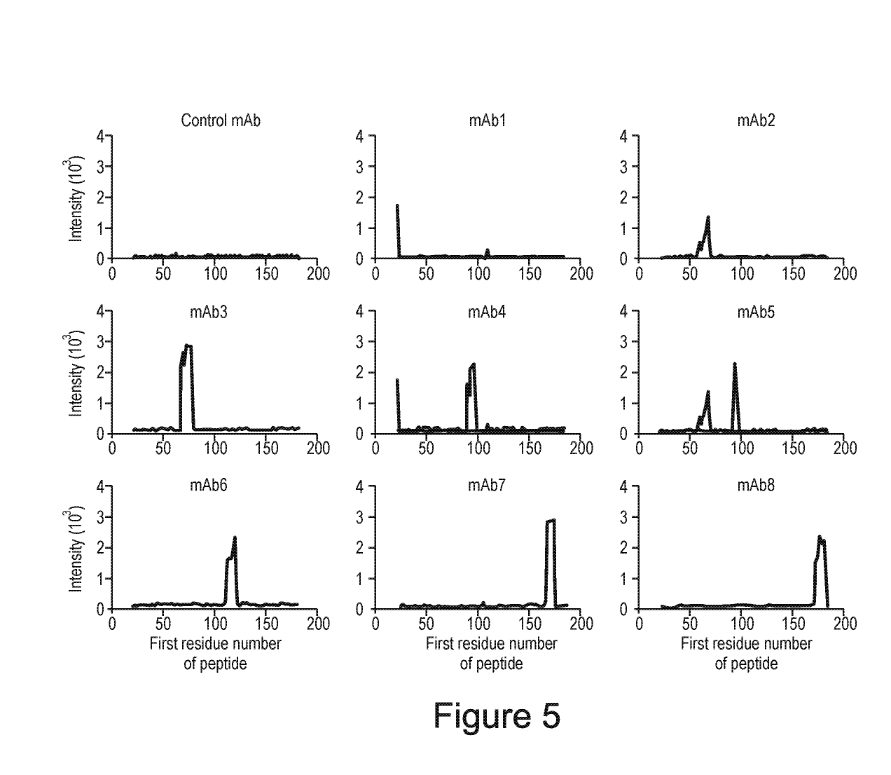

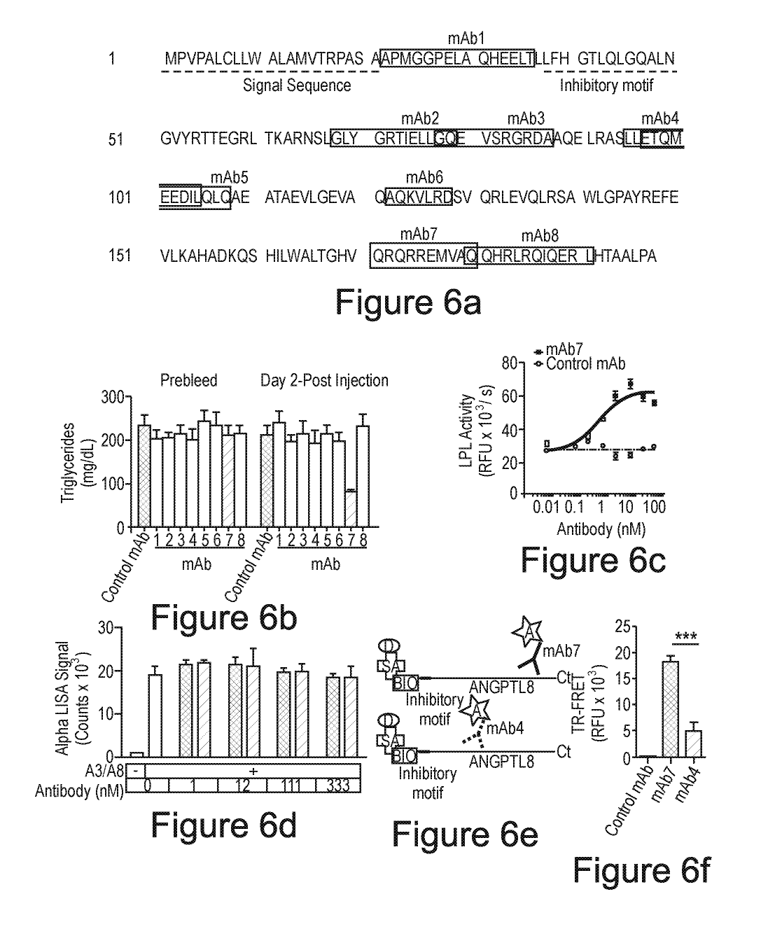

A library of peptides of 15 amino acids in length and with an offset of one residue was used to identify epitopes for 8 monoclonal antibodies that bind human ANGPTL8 with high affinity (K_{d} =2.4 ×10⁻¹⁰ to 5.8×10⁻⁹ M; FIG. 5). The epitopes are depicted in FIG. 6a. The antibodies do not cross react to mouse ANGPTL8 and were tested for invivo efficacy in humanized ANGPTL8 mice (Gusarova et al., Submitted 2017). Surprisingly, only one antibody (mAb 7 or H4H15341 P) produced a significant reduction in serum TG (FIG. 6b). This antibody binds to an epitope (amino acids 171-180) in the C-terminal region of ANGPTL8 (FIG. 6a). We have recently reported that this antibody also lowers circulating TG by 65% in cynomolgus monkeys (Gusarova et al., Submitted 2017). FIG. 6c shows that the antibody dose-dependently (EC₅₀=0.47 nM) reversed the inhibitory effect of ANGPTL8 on LPL. This study was performed in HEK293 cells co-expressing ANGPTL3.

Since the anti-ANGPTL8 blocking antibody binds to the C-terminal part and the LPL inhibitory motif is located in the N-terminal region, we wanted to test if it mediates its action by disrupting the interaction of ANGPTL3 and ANGPTL8. To this end, we used an AlphaLISA proximity assay where a signal is detected when ANGPTL3 and ANGPTL8 are expressed together (FIG. 6d, left). The AlphaLISA signal remained strong even when high concentrations of the antibody were tested. These data indicate that the anti-ANGPTL 8 blocking antibody does not restore LPL activity by disrupting the ANGPTL8:ANGPTL3 interaction.

Structural modeling has recently predicted that ANGPTL8 folds in a manner so that the N- and C-terminal domains are in close proximity (Siddiqa et al. (2016) Comput Biol Chem, 61, 210-220). Thus, we hypothesized that the ANGPTL8 blocking antibody may sterically shield the inhibitory motif in ANGPTL8 to prevent LPL inhibition. To test this hypothesis, we labeled the ANGPTL8 blocking antibody and a non-blocking antibody to ANGPTL8 (mAb4 (H4H15347P)), which binds to the mid region of ANGPTL8 protein sequence, with a FRET acceptor dye. In addition, we labeled ANGPTL8 in its N-terminal site with a donor dye as depicted in FIG. 6e. When labeled ANGPTL8 was co-expressed with ANGPTL3 in HEK293 cells and assayed with the labeled antibodies, we detected a much stronger TR-FRET signal with the ANGPTL8 blocking antibody directed to C-terminal fragment compared to that of the non-blocking antibody (FIG. 6f). These results indicate that the N- and C-termini of ANGPTL8 are located in close proximity and that the ANGPTL8 blocking antibody may sterically prevent binding of the ANGPTL8 inhibitory motif to LPL.

### Materials and Methods:

Epitope mapping. Epitope mapping was performed by PEPSCAN PRESTO BE. Briefly, standard Fmoc-peptide synthesis was used to synthesize 15-amino acid long peptides with 14 amino acid overlap. Antibody binding to each peptide was tested in a PEPSCAN-based ELISA: primary antibody was incubated at 1 μg/ml (overnight at 4° C.) followed by incubation with goat anti-human HRP conjugate (1 h at 25° C.). After washing, the peroxidase substrate 2,2′-azino-di-3ethylbenzthiazoline sulfonate (ABTS) and 10 μl/ml of 3% H₂O₂ were added and color development was measured using a charge couple device camera.

AlphaLISA assay. Assay was performed in a 384-well plate format at room temperature. CHO-K1 cell media (2.5 μl) from cells transfected with a vector containing ANGPTL3-myc and ANGPTL8-V5 or empty vector was incubated with the indicated amounts of antibodies for 1 h in a final volume of 20 μl. Then anti-V5 Alpha acceptor beads (Perkin Elmer) were added and incubated for an additional 30 min, follow by 1 h incubation with biotinylated anti-myc antibody (generated in house) and streptavidin Alpha donor beads (Perkin Elmer). All dilutions were performed in Hi-Block buffer (PerkinElmer), final concentration was 10 ng/ml for beads and 10 nM for biotinylated antibody in total volume of 50 μl. AlphaLISA signal was measured in an Envision® Multilabel Reader (PerkinElmer) following manufacturer's instructions.

TR-FRET antibody experiment. HEK293T cells were co-transfected with a plasmid coding for ANGPTL3 and a plasmid coding for N-terminal Avi-tagged ANGPTL8 using TranslT-LT1 transfection reagent. Cell media was collected after 72 h, concentrated 20× using Centriprep (Millipore) filter unit with a 10 kDa molecular cutoff, and site direct biotinylated using biotin-protein ligase BirA (Avidity) (Fairhead & Howarth, 2015) following supplier instructions, then dialyzed against PBS. Biotinylation of Avi-ANGPTL 8 was confirmed by Western blot using streptavidin-HRP for detection.

Antibodies were labeled using Alexa Fluor™ 647 Antibody Labeling Kit (Invitrogen). Antibody and dye concentrations were determined spectrophotometrically and drug to antibody ratios of 5:2 were used for all experiments. TR-FRET was performed in a 384-well plate format. Final concentration were 31.3 nM Europium-streptavidin, 25 nM labeled antibody, 50% Biotin-Avi-ANGPTL8 in TR-FRET dilution buffer (PerkinElmer). TR-FRET was measured using Envision® Multilabel Reader (PerkinElmer), excitation filter 340/30 nm, emission filter 1—615/8.5 nm, emission filter 2—665/7.5 nm, dichroic mirror D400/D630, measured in a delay of 100 ms and a window time of 2 ms.

We claim:

1. A method of treating a patient suffering from hyperlipidaemia, the method comprising administering to the patient a therapeutically effective amount of a combination of an angiopoietin-like protein 8 (ANGPTL8) inhibitor and an inhibitor of angiopoietin-like protein 3 (ANGPTL3).

2. The method of claim 1, wherein the hyperlipidaemia is familial hyperlipidaemia or acquired hyperlipidaemia.

3. The method of claim 1, wherein the hyperlipidaemia is selected from the group consisting of hyperlipoproteinemia, hypercholesterolemia, hypertriglyceridemia, and hyper chylomicronemia.

4. The method of claim 2, wherein the familial hyperlipidaemia is hypercholesterolemia selected from the group consisting of heterozygous familial hypercholesterolemia (HeFH) and homozygous familial hypercholesterolemia (HoFH).

5. The method of claim 2, wherein the acquired hyperlipidaemia is the result of excessive drinking of alcohol, obesity, side effects of medications (e.g. hormones or steroids), diabetes, kidney disease, underactive thyroid gland, or pregnancy.

6. The method of claim 1, wherein the ANGPTL8 inhibitor is an antibody, or an antigen-binding fragment thereof, that binds specifically to ANGPTL8.

7. The method of claim 6, wherein the antibody, or antigen-binding fragment thereof, that binds specifically to ANGPTL8 comprises the complementary determining regions (CDRs) of a heavy chain variable (HCVR) having the amino acid sequence of SEQ ID NO: 1 and the CDRs of a light chain variable region (LCVR) having the amino acid sequence of SEQ ID NO: 5.

8. The method of claim 6, wherein the antibody, or antigen-binding fragment thereof, that binds specifically to ANGPTL8 comprises a heavy chain CDR1 (HCDR1) having the amino acid sequence of SEQ ID NO: 2, a HCDR2 having the amino acid sequence of SEQ ID NO: 3, a HCDR3 having the amino acid sequence of SEQ ID NO: 4, a light chain CDR1 (LCDR1) having the amino acid sequence of SEQ ID NO: 6, a LCDR2 having the amino acid sequence of SEQ ID NO: 7, and a LCDR3 having the amino acid sequence of SEQ ID NO: 8.

9. The method of claim 6, wherein the antibody, or antigen-binding fragment thereof, that binds specifically to ANGPTL8 comprises a HCVR having the amino acid sequence of SEQ ID NO: 1 and a LCVR having the amino acid sequence of SEQ ID NO: 5.

10. The method of claim 1, wherein the ANGPTL8 inhibitor is administered to the patient subcutaneously or intravenously.

11. The method of claim 1, wherein the ANGPTL3 inhibitor is an antibody, or an antigen-binding fragment thereof, that binds specifically to ANGPTL3.

12. The method of claim 11, wherein the ANGPTL3 antibody is evinacumab.

13. The method of claim 11, wherein the antibody, or antigen-binding fragment thereof, that binds specifically to ANGPTL3 comprises the complementary determining regions (CDRs) of a heavy chain variable (HCVR) having the amino acid sequence of SEQ ID NO: 10 and the CDRs of a light chain variable region (LCVR) having the amino acid sequence of SEQ ID NO: 14.

14. The method of claim 11, wherein the antibody, or antigen-binding fragment thereof, that binds specifically to ANGPTL3 comprises a heavy chain CDR1 (HCDR1) having the amino acid sequence of SEQ ID NO: 11, a HCDR2 having the amino acid sequence of SEQ ID NO: 12, a HCDR3 having the amino acid sequence of SEQ ID NO: 13, a light chain CDR1 (LCDR1) having the amino acid sequence of SEQ ID NO: 15, a LCDR2 having the amino acid sequence of SEQ ID NO: 16, and a LCDR3 having the amino acid sequence of SEQ ID NO: 17.

15. The method of claim 11, wherein the antibody, or antigen-binding fragment thereof, that binds specifically to ANGPTL3 comprises a HCVR having the amino acid sequence of SEQ ID NO: 10 and a LCVR having the amino acid sequence of SEQ ID NO: 14.

16. The method of claim 11, wherein the ANGPTL3 antibody, or antigen-binding fragment thereof, is administered to the patient subcutaneously or intravenously.

17. A method of reducing the level of at least one lipid parameter in a patient suffering from a disorder or condition characterized in part by elevated levels of lipids or lipoproteins, the method comprising administering to the patient a therapeutically effective amount of a combination of an angiopoietin-like protein 8 (ANGPTL8) inhibitor and an inhibitor of angiopoietin-like protein 3 (ANGPTL3).

18. The method of claim 17, wherein the disorder or condition is hyperlipidaemia selected from the group consisting of familial hyperlipidaemia and acquired hyperlipidaemia.

19. The method of claim 17, wherein the disorder or condition is hyperlipidaemia selected from the group consisting of hyperlipoproteinemia, hypercholesterolemia, hypertriglyceridemia, and hyper chylomicronemia.

20. The method of claim 18, wherein the familial hyperlipidaemia is hypercholesterolemia selected from the group consisting of heterozygous familial hypercholesterolemia (HeFH) and homozygous familial hypercholesterolemia (HoFH).

21. The method of claim 18, wherein the acquired hyperlipidaemia is the result of excessive drinking of alcohol, obesity, side effects of medications (e.g. hormones or steroids), diabetes, kidney disease, underactive thyroid gland, or pregnancy.

22. The method of claim 17, wherein the ANGPTL8 inhibitor is an antibody, or an antigen-binding fragment thereof, that binds specifically to ANGPTL8.

23. The method of claim 22, wherein the antibody, or antigen-binding fragment thereof, that binds specifically to ANGPTL8 comprises the complementary determining regions (CDRs) of a heavy chain variable (HCVR) having the amino acid sequence of SEQ ID NO: 1 and the CDRs of a light chain variable region (LCVR) having the amino acid sequence of SEQ ID NO: 5.

24. The method of claim 22, wherein the antibody, or antigen-binding fragment thereof, that binds specifically to ANGPTL8 comprises a heavy chain CDR1 (HCDR1) having the amino acid sequence of SEQ ID NO: 2, a HCDR2 having the amino acid sequence of SEQ ID NO: 3, a HCDR3 having the amino acid sequence of SEQ ID NO: 4, a light chain CDR1 (LCDR1) having the amino acid sequence of SEQ ID NO: 6, a LCDR2 having the amino acid sequence of SEQ ID NO: 7, and a LCDR3 having the amino acid sequence of SEQ ID NO: 8.

25. The method of claim 22, wherein the antibody, or antigen-binding fragment thereof, that binds specifically to ANGPTL8 comprises a HCVR having the amino acid sequence of SEQ ID NO: 1 and a LCVR having the amino acid sequence of SEQ ID NO: 5.

26. The method of claim 22, wherein the ANGPTL8 antibody, or antigen-binding fragment thereof, is administered to the patient subcutaneously or intravenously.

27. The method of claim 17, wherein the ANGPTL3 inhibitor is an antibody, or an antigen-binding fragment thereof, that binds specifically to ANGPTL3.

28. The method of claim 27, wherein the ANGPTL3 antibody is evinacumab.

29. The method of claim 27, wherein the antibody, or antigen-binding fragment thereof, that binds specifically to ANGPTL3 comprises the complementary determining regions (CDRs) of a heavy chain variable (HCVR) having the amino acid sequence of SEQ ID NO: 10 and the CDRs of a light chain variable region (LCVR) having the amino acid sequence of SEQ ID NO: 14.

30. The method of claim 27, wherein the antibody, or antigen-binding fragment thereof, that binds specifically to ANGPTL3 comprises a heavy chain CDR1 (HCDR1) having the amino acid sequence of SEQ ID NO: 11, a HCDR2 having the amino acid sequence of SEQ ID NO: 12, a HCDR3 having the amino acid sequence of SEQ ID NO: 13, a light chain CDR1 (LCDR1) having the amino acid sequence of SEQ ID NO: 15, a LCDR2 having the amino acid sequence of SEQ ID NO: 16, and a LCDR3 having the amino acid sequence of SEQ ID NO: 17.

31. The method of claim 27, wherein the antibody, or antigen-binding fragment thereof, that binds specifically to ANGPTL3 comprises a HCVR having the amino acid sequence of SEQ ID NO: 10 and a LCVR having the amino acid sequence of SEQ ID NO: 14.

32. The method of claim 27, wherein the ANGPTL3 antibody, or antigen-binding fragment thereof, is administered to the patient subcutaneously or intravenously.

33. The method of claim 1, wherein the ANGPTL8 inhibitor and the ANGPTL3 inhibitor are administered concurrently or sequentially.

34. The method of claim 1, wherein the ANGPTL8 inhibitor and the ANGPTL3 inhibitor are administered at therapeutically effective concentrations in separate pharmaceutical compositions or are co-formulated in one pharmaceutical composition.
